(19) United States
(12) Patent Application Publication
(10) Pub. No.: US 2024/0228618 A1
(43) Pub. Date: Jul. 11, 2024

(54) COMPOSITIONS COMPRISING A T CELL REDIRECTION THERAPEUTIC AND AN ANTI-CD44 THERAPEUTIC

(71) Applicant: Janssen Biotech, Inc., Horsham, PA (US)

(72) Inventors: Bradley J. Heidrich, Gilberstville, PA (US); Priyanka Nair-Gupta, Doylestown (UA); Francois Gaudet, Princeton, NJ (US)

(21) Appl. No.: 18/561,512

(22) PCT Filed: May 16, 2022
(86) PCT No.: PCT/IB2022/054529

§ 371 Date: Nov. 16, 2023

Related U.S. Application Data
(60) Provisional application No. 63/189,737, filed on May 18, 2021.

(51) Int. Cl. C07K 16/28; A61K 39/00; A61P 35/02

(52) U.S. Cl. CPC C07K 16/2809; A61P 35/02; C07K 16/2842; C07K 16/2866; C07K 16/2884; A61K 2039/507; A61K 2039/545; C07K 2317/31; C07K 2317/56; C07K 2317/76

(57) ABSTRACT

Disclosed herein is a pharmaceutical composition comprising a T cell redirect therapeutic and an anti-CD44 therapeutic, and uses thereof for killing cancer cells.

### CROSS REFERENCE TO RELATED APPLICATION

This application claims the benefit of U.S. Provisional Application 63/189,737, filed on May 18, 2021, which is incorporated by reference herein in its entirety.

### FIELD OF THE INVENTION

This disclosure relates to compositions and cancer treatments utilizing T cell redirection therapeutics.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created on Feb. 28, 2022, is named JBI6553WOPCT1_SeqListing.txt and is 24,576 bytes in size.

### BACKGROUND OF THE INVENTION

Despite several treatment options, there is currently no cure for acute myeloid leukemia (AML) and multiple myeloma (MM). Even after achieving high rates (50%-80%) of complete hematologic remission (CR), defined as the presence of ≤5% of leukemic blasts (AML) or plasma cells (MM) in the bone marrow (BM) (1, 2), the majority of patients with AML or MM relapse (3-5). Relapse has been linked to minimal residual disease (MRD) whereby small numbers of cancer stem cells (CSC), or other malignant progenitor cells, fail to be cleared and persist even after therapy (6). Preventing relapses and finding cures for AML and MM requires finding better strategies to eliminate MRD.

Like hematopoietic stem cells (HSC), CSC in AML and MM reside and preferentially persist in the BM niche (7, 8). The BM niche provides a specialized microenvironment via secretion of soluble growth factors and cell-cell interactions that are protective to the CSC (9). Moreover, the BM niche is immune-suppressive and is appreciated to be a site of immune privilege at steady state to allow for normal hematopoiesis and immune cell generation (10). These aspects of the BM niche have provided resistance against and minimized the efficacy of several anti-cancer drugs including chemotherapy, targeted small molecule inhibitors, and antibody based therapies (11-14).

The ability of T cells to specifically lyse tumor cells and secrete cytokines to recruit and support immunity against cancer makes them an attractive option for therapy. Several approaches have capitalized on this strategy such as bispecific T-cell engagers (small bispecific biologics), chimeric antigen receptors (CARs) and bispecific antibodies, among others (15). Bispecific T-cell engagers and antibody-mediated redirection cross-link T cells to tumor cells by engaging a specific epitope on tumor cells and CD3 on T cells, leading to T cell activation, and secretion of perforins and granzymes that ultimately kill the tumor cells. These CD3 redirection therapies have been validated as an effective anti-cancer strategy in the clinic with the approval of CD19×CD3 (blinatumomab) for acute lymphoblastic lymphoma (ALL) (16). However, the immunosuppressive and protective nature of the BM niche potentially poses a significant hurdle to T cell redirection therapies.

For example, as shown herein, using bispecific antibodies targeting specific tumor antigens (CD123) and CD3, it was observed that co-culture of AML cell lines with BM stromal cells significantly protected cancer cells from bispecific-T cell-mediated lysis in vitro. Impaired CD3 redirection cytotoxicity was correlated with reduced T cell effector responses, thereby providing a mechanism to explain loss of activity of the bispecific antibody.

### BRIEF SUMMARY OF THE INVENTION

Provided herein is pharmaceutical composition comprising a T cell redirection therapeutic and an anti-CD44 therapeutic, wherein, the T cell redirection therapeutic comprises a first binding region that immunospecifically binds a T cell surface antigen and a second binding region that immunospecifically binds a tumor associated antigen (TAA).

In one embodiment of the pharmaceutical composition, the composition further comprises a pharmaceutically acceptable carrier.

In a further embodiment of the pharmaceutical composition, the T cell redirection therapeutic is an antibody or antigen-binding fragment thereof.

In a yet further embodiment of the pharmaceutical composition, the T cell surface antigen is selected from the group consisting of CD3, CD2, CD4, CD5, CD6, CD8, CD28, CD40L, CD44, KI2L4, NKG2E, NKG2D, NKG2F, BTNL3, CD186, BTNL8, PD-1, CD195, and NKG2C.

In a yet further embodiment of the pharmaceutical composition, the T cell surface antigen is CD3.

In a yet further embodiment of the pharmaceutical composition, the TAA is selected from the group consisting of CD123, BCMA, GPRC5D, CD33, CD19, PSMA, TMEFF2, CD20, CD22, CD25, CD52, ROR1, HM1.24, CD38, and SLAMF7.

In a yet further embodiment of the pharmaceutical composition, the T cell redirection therapeutic is a CD123×CD3 bispecific antibody having a first antigen-binding site that immunospecifically binds CD123 and a second antigen-binding site that immunospecifically binds CD3. In one embodiment, the CD123×CD3 bispecific antibody comprises a first heavy chain (HC1), a first light chain (LC1), a second heavy chain (HC2), and a second light chain (LC2), and wherein the HC1 and the LC1 pair to form the first antigen-binding site and the HC2 and the LC2 pair to form the second antigen-binding site. In one embodiment, the HC1 comprises the amino acid sequence of SEQ ID NO: 1, the LC1 comprises the amino acid sequence of SEQ ID NO: 2, the HC2 comprises the amino acid sequence of SEQ ID NO: 3, and the LC2 comprises the amino acid sequence of SEQ ID NO: 4.

In a yet further embodiment of the pharmaceutical composition, the T cell redirection therapeutic is a BCMA×CD3 bispecific antibody having a first antigen-binding site that immunospecifically binds CD123 and a second antigen-binding site that immunospecifically binds CD3. In one embodiment, the BCMA×CD3 bispecific antibody comprises a first heavy chain (HC1), a first light chain (LC1), a second heavy chain (HC2), and a second light chain (LC2), and wherein the HC1 and the LC1 pair to form the first antigen-binding site and the HC2 and the LC2 pair to form the second antigen-binding site. In one embodiment, the HC1 comprises the amino acid sequence of SEQ ID NO: 5, the LC1 comprises the amino acid sequence of SEQ ID NO: 6, the HC2 comprises the amino acid sequence of SEQ ID NO: 7, and the LC2 comprises the amino acid sequence of SEQ ID NO: 8.

In a yet further embodiment of the pharmaceutical composition, the anti-CD44 therapeutic is an anti-CD44 antibody or antigen-binding fragment thereof.

In a yet further embodiment of the pharmaceutical composition, the anti-CD44 antibody or antigen-binding fragment thereof is selected from the group consisting of monoclonal antibodies, scFv, Fab, Fab′, F(ab′)2, and F(v) fragments, heavy chain monomers or dimers, light chain monomers or dimers, and dimers consisting of one heavy chain and one light chain.

In a yet further embodiment of the pharmaceutical composition, the anti-CD44 therapeutic is a CD44 antagonist.

In a yet further embodiment of the pharmaceutical composition, the composition further comprises a VLA-4 adhesion pathway inhibitor.

In a yet further embodiment of the pharmaceutical composition, the VLA-4 adhesion pathway inhibitor is an anti-VLA-4 antibody or antigen-binding fragment thereof.

In a yet further embodiment of the pharmaceutical composition, the anti-VLA-4 antibody or antigen-binding fragment thereof is selected from the group consisting of monoclonal antibodies, scFv, Fab, Fab′, F(ab′)2, and F(v) fragments, heavy chain monomers or dimers, light chain monomers or dimers, and dimers consisting of one heavy chain and one light chain.

In a yet further embodiment of the pharmaceutical composition, the VLA-4 adhesion pathway inhibitor is a VLA-4 antagonist.

In a yet further embodiment of the pharmaceutical composition, the VLA-4 antagonist is selected from the group consisting of BIO1211, TCS2314, BIO5192, and TR14035.

Further provided herein is a method of treating cancer in a subject in need thereof, comprising administering to the subject a therapeutically effective amount of the pharmaceutical composition as provided above.

In one embodiment of the method, the subject has a newly diagnosed cancer.

In a further embodiment of the method, the subject is relapsed or refractory to a prior anti-cancer therapy.

In a yet further embodiment of the method, the cancer is a hematological malignancy or a solid tumor.

In a yet further embodiment of the method, the subject has AML or MM.

In a yet further embodiment of the method, the T cell redirection therapeutic and the anti-CD44 therapeutic are administered simultaneously or sequentially.

In a yet further embodiment of the method, the anti-CD44 therapeutic is administered prior to administration of the T cell redirection therapeutic.

In a yet further embodiment of the method, the anti-CD44 therapeutic is administered after administration of the T cell redirection therapeutic.

In a yet further embodiment of the method, the T cell redirection therapeutic and the VLA-4 adhesion pathway inhibitor are administered simultaneously or sequentially.

In a yet further embodiment of the method, the VLA-4 adhesion pathway inhibitor is administered prior to administration of the T cell redirection therapeutic.

In a yet further embodiment of the method, the VLA-4 adhesion pathway inhibitor is administered after administration of the T cell redirection therapeutic.

In a yet further embodiment of the method, the anti-CD44 therapeutic and the VLA-4 adhesion pathway inhibitor are administered simultaneously or sequentially.

In a yet further embodiment of the method, the anti-CD44 therapeutic is administered prior to administration of the VLA-4 adhesion pathway inhibitor.

In a yet further embodiment of the method, the anti-CD44 therapeutic is administered after administration of the VLA-4 adhesion pathway inhibitor.

Yet further provided herein is a kit comprising the pharmaceutical composition as provided above.

### BRIEF DESCRIPTION OF THE DRAWINGS

The foregoing summary, as well as the detailed description and embodiments of the present application, will be better understood when read in conjunction with the appended claims and drawings. It should be understood, however, that the invention is not limited to the precise recitations disclosed herein.

FIG. 1A and FIG. 1B are graphs showing the cytotoxicity and T cell activation rates when human T cells (8×10⁴ cells/well) were co-cultured with CFSE-labeled KG-1 or HL-60 (4×10⁴ cells/well) in the absence (square) or presence (black circle) of stromal cells (4×10⁴ cells/well). The CD123×CD3 bispecific antibody was added for 48 hours in a dose response (24 h after co-culture), starting at 100 nM (15 μg/mL) and diluted 10-fold to the conditions for each subsequent dose. Cytotoxicity (FIG. 1A) was evaluated by calculating percentage of dead CFSE⁺ target cells, which was quantified by flow cytometry. T cell activation (FIG. 1B) was evaluated as a percentage of live CD45⁺/CD3⁺/CD25⁺ T cells. The dose titrations were plotted from 3 individual T cell donors and analyzed with a 4-parameter nonlinear regression curve fit. T cells from 3 healthy donors were tested in T-cell redirection assays with the indicated cell lines. Mean±SD was graphed and were representative of two experiments. MSC: mesenchymal stem cells.

FIG. 2 are bar graphs showing the changes of secreted cytokines mediated by the CD123×CD3 bispecific antibody when BM stromal cells were co-cultured with KG-1 cells. Supernatants from a 72-hour co-culture assay with KG-1 cells were evaluated for human cytokines IL-1β (a.), IL-1ra (b.), IL-2 (c.), IL-3 (d.), TNF-α (e.), IFN-γ (f.) and chemokines G-CSF (g.) and VEGF (h.) in a MILLIOPLEX® MAP Kit. Conditions included KG-1 cells+T cells (“No Stroma”) or KG-1 cells+T cells+BM stromal cells (“BM Stroma”). Supernatants were pooled for each T cell donor in duplicate from two experiments and graphed as mean±SD.

FIG. 3 are bar graphs showing the changes of secreted cytokines mediated by the CD123×CD3 bispecific antibody when BM stromal cell were co-cultured with HL-60 cells. Supernatants from a 72-hour co-culture assay with HL-60 cells were evaluated for human cytokines IL-1β (a.), IL-1ra (b.), IL-2 (c.), IL-3 (d.), TNF-α (e.), IFN-γ (f.) and chemokines G-CSF (g.) and VEGF (h.) in a MILLIOPLEX® MAP Kit. Conditions included KG-1 cells+T cells (“No Stroma”) or KG-1 cells+T cells+BM stromal cells (“BM Stroma”). Supernatants were pooled for each T cell donor in duplicate from two experiments and graphed as mean±SD.

FIG. 4A and FIG. 4B are graphs showing that CD44 is expressed on AML and HS-5 stromal cells and significantly increased on primary AML patient samples. (FIG. 4A) AML cell lines and HS-5 stromal cells were evaluated by flow cytometry for cell surface markers to confirm CD44, CD123, VLA-4, VLA-5, CXCR4, FLT3 and the CD3 antigen was used as a negative control. Top: Unstained; Middle: Isotype; Bottom: Stained. (FIG. 4B) Bioinformatic analysis of public data set (GSE15061) shows expression on AML, MDS, and non-leukemia bone marrow samples (normal). mRNA expression was plotted on log 2 scale on the y-axis (AML, n=202; MDS, n=164, normal, n=69). One-way ANOVA with Tukey multiple comparison test (alpha=0.05) was performed to compare the groups, **** p<0.0001; ns, not significantly different. FIG. 4A is representative of 3 experimental repeats.

FIG. 5 are graphs showing that the stromal-mediated suppression of killing by the CD123×CD3 bispecific antibody was restored by the anti-CD44 antibody. Human T cells (80,000 cells/well) were co-cultured with CFSE-labeled AML (KG-1, HL-60, MV4-11, MOLM-13, SKNO-1) and HS-5 stromal cells (40,000 cells/well; black line) with anti-CD44 (20 μg/mL). After 24 hours, the CD123×CD3 bispecific antibody was added in a dose response, starting at 100 nM (15 μg/mL) and diluted 10-fold for the subsequent doses for an additional 48 hours. Cytotoxicity was evaluated by calculating percentage of dead CFSE⁺ target cells; T cell activation was evaluated as a percentage of CD25⁺ and PD-1⁺ T cells, which was quantified by flow cytometry. T cells from 3 healthy donors were tested in T-cell redirection assays with the indicated cell lines. Mean±SD was graphed and were a representative graph of at least three or more experiments. NS=no stroma.

FIG. 6 are graphs showing that CD44, VLA-4, and CXCR-4 are expressed on KG-1 cells, T cells, and stromal cells. KG-1 cells, T cells, primary bone marrow (BM) stromal cells, and HS-5 stromal cells were evaluated by flow cytometry for cell surface markers to evaluate CD44, VLA-4, and CXCR4 expression. Stromal cell lines expressed very high levels of CD44 with moderately lower levels on KG-1 and T cells. VLA-4 was observed at uniform levels among all the cells tested. CXCR4 was expressed at lower levels on KG-1 cells and higher levels on T cells and stromal cells. Data shown were representative of 2 experimental repeats. Top: Isotype; Bottom: Stained.

FIG. 7 are graphs showing that HS-5 stromal mediated suppression of the CD123×CD3 bispecific antibody in co-culture with KG-1 cells was restored by the anti-CD44 antibody. Supernatants from a 72-hour co-culture assay were evaluated for human cytokines IL-2, IL-3, TNF-α, IFN-γ in a MILLIOPLEX® MAP Kit. Conditions included KG-1 cells+T cells (no stroma)+/−anti-CD44 antibody or KG-1 cells+T cells+HS-5 stromal cells+/−anti-CD44 antibody, or cells alone. Supernatants were pooled for each T cell donor in duplicate from two experiments and graphed as mean±SD.

FIG. 8 are graphs showing that CD44 is not detectable in KG-1 and HS-5 CD44 KO cell lines. CD44 was knocked out of the KG-1 and HS-5 stromal cell lines and generated by limiting dilutions to make clonal populations. The cells were evaluated by flow cytometry for cell surface markers to confirm knockout of CD44 and that other cell surface markers, CD123, VLA-4, VLA-5, CXCR4, and FLT3 were unchanged. The CD3 antigen was used as a negative control. Data were representative of 3 experiments. Top: Unstained; Middle: Isotype; Bottom: Stained.

FIG. 9 are graphs showing that blockade of CD44 in KG-1 and HS-5 cells restored the stromal-mediated suppression of cytotoxicity and T cell activation with the CD123×CD3 bispecific antibody. Human T cells (8×10⁴ cells/well) were co-cultured with CFSE-labeled AML (KG-1^{wt} or KG-1^{CD44KO}) in the absence or presence of HS-5^{wt} or HS-5^{CD44KO} stromal cells (4×10⁴ cells/well) with the anti-CD44 antibody (20 μg/mL). After 24 hours, the CD123×CD3 bispecific antibody was added at 0.001 nM (0.045 μg/mL) for an additional 24 hours. Cytotoxicity was evaluated by calculating percentage of dead CFSE⁺ target cells; T cell activation was evaluated as a percentage of CD25⁺ T cells, which was quantified by flow cytometry. Mean±SD was graphed and were a representative graph of two experiments. KG-1 cells cultured with HS-5 cells demonstrate the maximum suppression observed in the experiment. T cells from 3 healthy donors were tested in T-cell redirection assays with the indicated cell lines. Mean±SD was graphed and were a representative graph of two experiments.

FIG. 10 are graphs showing that blockade of CD44 enhanced granzyme-B levels and increased PD-1 expression. Human T cells (8×10⁴ cells/well) were co-cultured with CFSE-labeled AML (KG-1^{wt} or KG-1^{CD44KO}) and HS-5^{wt} or HS-5^{CD44KO} stromal cells (4×10⁴ cells/well) with anti-CD44 (20 μg/mL). After 24 hours, CD123×CD3 was added at 0.001 nM (0.045 μg/mL) for an additional 24 hours. Granzyme-B levels in T cells as well as T cell early activation was evaluated as a percentage of PD-1⁺ T cells, which was quantified by flow cytometry. T cells from 3 healthy donors were tested in T-cell redirection assays with the indicated cell lines. Mean±SD was graphed and were a representative graph of at least two or more experiments.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

The words “right,” “left,” “lower,” and “upper” designate directions in the drawings to which reference is made.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

As used herein, the term “isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if the composition is not part of the native environment of the nucleic acid, peptide, or protein. The term also embraces nucleic acids, peptides and proteins prepared by recombinant expression in a host cell as well as chemically synthesized nucleic acids.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected or transduced with a nucleic acid molecule of the invention. In another embodiment, a “host cell” is a progeny or potential progeny of such a transfected or transduced cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications.

The term “antibodies” as used herein, is meant in a broad sense and includes immunoglobulin molecules including monoclonal antibodies (including murine, human, humanized and chimeric monoclonal antibodies), antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g. IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4 Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

The term “monoclonal antibody” as used herein, refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies may have heterogeneous glycosylation within the antibody population. Monoclonal antibody may be monospecific or multispecific such as bispecific, monovalent, bivalent or multivalent.

The term “human antibody” as used herein, refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

The term “humanized antibody” as used herein, refers to an antibody in which at least one CDR is derived from non-human species and at least one framework is derived from human immunoglobulin sequences. Humanized antibody may include substitutions in the frameworks so that the frameworks may not be exact copies of expressed human immunoglobulin or human immunoglobulin germline gene sequences.

The term “isolated antibody” refers to an antibody that is substantially free of other cellular material and/or chemicals and encompasses antibodies that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

The term “antigen binding fragment” or “antigen binding domain” as used herein, refers to a portion of an immunoglobulin molecule that binds an antigen. Antigen binding fragments may be synthetic, enzymatically obtainable or genetically engineered polypeptides and include the VH, the VL, the VH and the VL, Fab, F(ab′)2, Fd and Fv fragments, domain antibodies (dAb) consisting of one VH domain or one VL domain, shark variable IgNAR domains, camelized VH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3. VH and VL domains may be linked together via a synthetic linker to form various types of single chain antibody designs where the VH/VL domains may pair intramolecularly, or intermolecularly in those cases when the VH and VL domains are expressed by separate single chain antibody constructs, to form a monovalent antigen binding site, such as single chain Fv (scFv) or diabody; described for example in Int. Patent Publ. Nos. WO1998/44001, WO1988/01649, WO1994/13804 and WO1992/01047.

The term “bispecific” refers to an antibody that specifically binds two distinct antigens or two distinct epitopes within the same antigen. The bispecific antibody may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

The term “multispecific” as used herein, refers to an antibody that specifically binds at least two distinct antigens or at least two distinct epitopes within the same antigen. Multispecific antibody may bind for example two, three, four or five distinct antigens or distinct epitopes within the same antigen.

“Specific binding” or “immunospecific binding” or derivatives thereof when used in the context of antibodies, or antibody fragments, represents binding via domains encoded by immunoglobulin genes or fragments of immunoglobulin genes to one or more epitopes of a protein of interest, without preferentially binding other molecules in a sample containing a mixed population of molecules. Typically, an antibody binds to a cognate antigen with a K_{d} of less than about 1×10⁻⁸ M, as measured by a surface plasmon resonance assay or a cell binding assay.

The term “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor.

The term “combination” as used herein, means that two or more therapeutics are administered to a subject together in a mixture, concurrently as single agents or sequentially as single agents in any order.

The term “enhance” or “enhanced” as used herein, refers to enhancement in one or more functions of a test molecule when compared to a control molecule or a combination of test molecules when compared to one or more control molecules. Exemplary functions that can be measured are tumor cell killing, T cell activation, relative or absolute T cell number, Fc-mediated effector function (e.g. ADCC, CDC and/or ADCP) or binding to an Fcγ receptor (FcγR) or FcRn. “Enhanced” may be an enhancement of about 10%, 20%, 30%, 40%, 50%, 60%, 70%, 80%, 90%, 100% or more, or a statistically significant enhancement.

The term “mutation” as used herein, refers to an engineered or naturally occurring alteration in a polypeptide or polynucleotide sequence when compared to a reference sequence. The alteration may be a substitution, insertion or deletion of one or more amino acids or polynucleotides.

The term “non-fixed combination” as used herein, refers to separate pharmaceutical compositions of the T cell redirection therapeutic and the VLA-4 adhesion pathway inhibitor administered as separate entities either simultaneously, concurrently or sequentially with no specific intervening time limits, wherein such administration provides effective levels of the two compounds in the body of the subject.

The term “pharmaceutical composition” as used herein, refers to composition that comprises an active ingredient and a pharmaceutically acceptable carrier.

The term “pharmaceutically acceptable carrier” or “excipient” as used herein, refers to an ingredient in a pharmaceutical composition, other than the active ingredient, which is nontoxic to a subject.

The term “recombinant” as used herein, refers to DNA, antibodies and other proteins that are prepared, expressed, created or isolated by recombinant means when segments from different sources are joined to produce recombinant DNA, antibodies or proteins.

The term “reduce” or “reduced” as used herein, refers to a reduction in one or more functions of a test molecule when compared to a control molecule or a combination of test molecules when compared to one or more control molecules. Exemplary functions that can be measured are tumor cell killing, T cell activation, relative or absolute T cell number, Fc-mediated effector function (e.g. ADCC, CDC and/or ADCP) or binding to an Fcγ receptor (FcγR) or FcRn. “Reduced” may be a reduction of about 10%, 20%, 30%, 40%, 50%, 60%, 70%, 80%, 90%, 100% or more, or a statistically significant enhancement.

The term “refractory” as used herein, refers to a cancer that is not amendable to surgical intervention and is initially unresponsive to therapy.

The term “relapsed” as used herein, refers to a cancer that responded to treatment but then returns.

The term “subject” as used herein, includes any human or nonhuman animal “Nonhuman animal” includes all vertebrates, e.g., mammals and non-mammals, such as nonhuman primates, sheep, dogs, cats, horses, cows, chickens, amphibians, reptiles, etc. Except when noted, the terms “patient” or “subject” are used interchangeably.

The term “therapeutically effective amount” as used herein, refers to an amount effective, at doses and for periods of time necessary, to achieve a desired therapeutic result. A therapeutically effective amount may vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient.

The term “treat” or “treatment” as used herein, refers to both therapeutic treatment and prophylactic or preventative measures, wherein the object is to prevent or slow down (lessen) an undesired physiological change or disorder. Beneficial or desired clinical results include alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if a subject was not receiving treatment. Those in need of treatment include those already with the condition or disorder as well as those prone to have the condition or disorder or those in which the condition or disorder is to be prevented.

The term “tumor cell” or a “cancer cell” as used herein, refers to a cancerous, pre-cancerous or transformed cell, either in vivo, ex vivo, or in tissue culture, that has spontaneous or induced phenotypic changes. These changes do not necessarily involve the uptake of new genetic material. Although transformation may arise from infection with a transforming virus and incorporation of new genomic nucleic acid, uptake of exogenous nucleic acid or it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

### T Cell Redirection Therapeutics

The T cell redirection therapeutic (which is also referred to as “T cell redirection bispecific antibody” or “the bispecific antibody” throughout this application) disclosed herein is a molecule containing two or more binding regions, wherein one of the binding regions specifically binds a cell surface antigen (such as a tumor associated antigen (TAA)) on a target cell or tissue and wherein a second binding region of the molecule specifically binds a T cell surface antigen (such as, CD3). This dual/multi-target binding ability recruits T cells to the target cell or tissue leading to the eradication of the target cell or tissue.

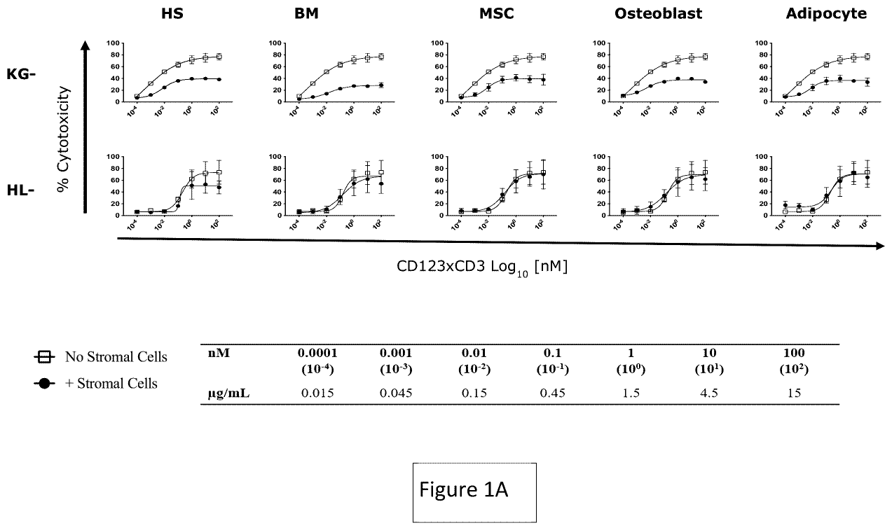

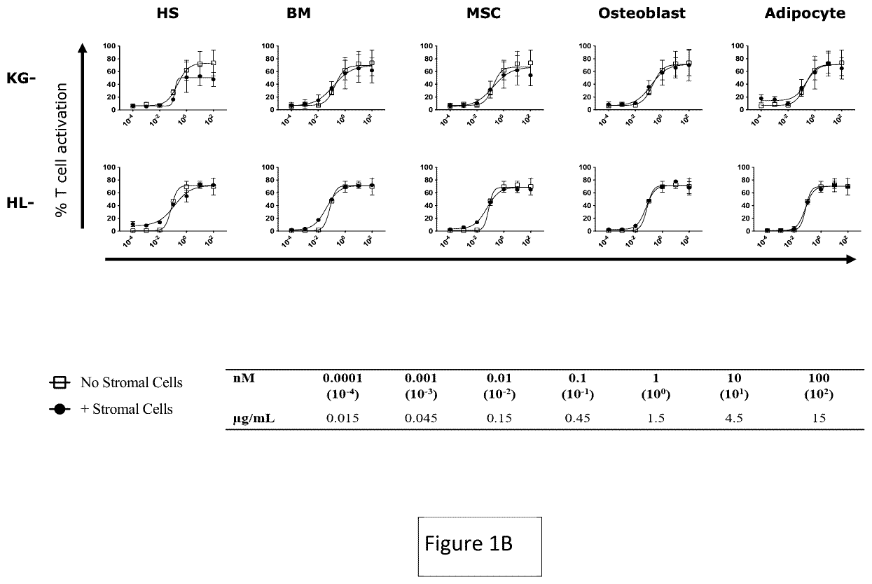

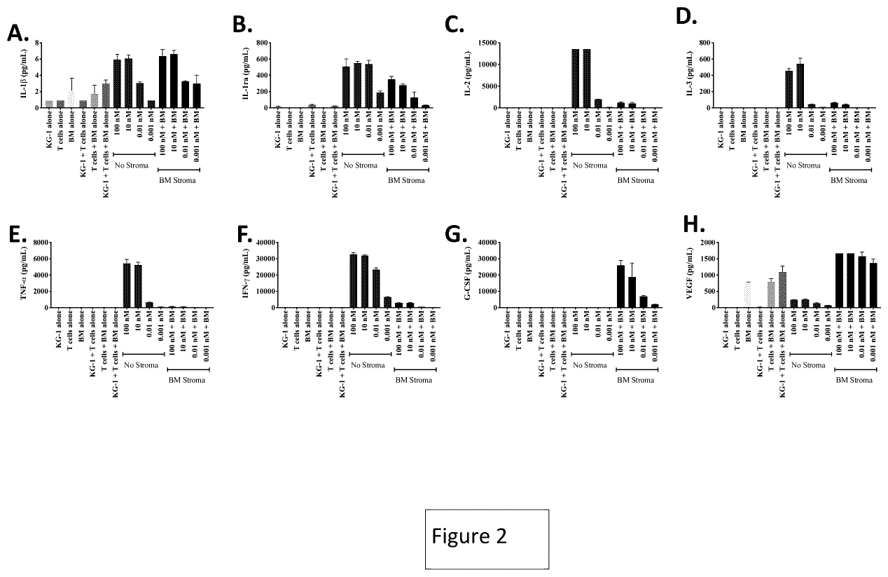

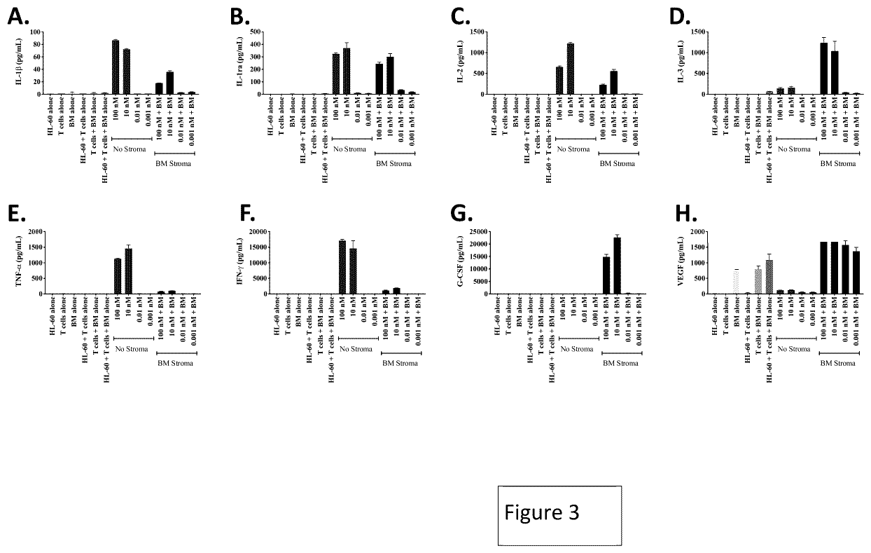

The T cell redirection therapeutic used herein may be an antibody, an antibody-derived protein, or, for example, a recombinant protein exhibiting antigen binding sites. In one embodiment, the T cell redirection therapeutics used herein are bispecific antibodies encompass “whole” antibodies, such as whole IgG or IgG-like molecules, and small recombinant formats, such as tandem single chain variable fragment molecules (taFvs), diabodies (Dbs), single chain diabodies (scDbs) and various other derivatives of these (cf. bispecific antibody formats as described by Byrne H. et al. (2013) Trends Biotech, 31 (11): 621-632 with FIG. 2 showing various bispecific antibody formats; Weidle U. H. et al. (2013) Cancer Genomics and Proteomics 10: 1-18, in particular FIG. 1 showing various bispecific antibody formats; and Chan, A. C. and Carter, P. J. (2010) Nat Rev Immu 10: 301-316 with FIG. 3 showing various bispecific antibody formats). Examples of bispecific antibody formats include, but are not limited to, quadroma, chemically coupled Fab (fragment antigen binding), and BiTE® (bispecific T cell engager).

In one embodiment, the bispecific antibody used herein may be selected from the group comprising Triomabs; hybrid hybridoma (quadroma); Multispecific anticalin platform (Pieris); Diabodies; Single chain diabodies; Tandem single chain Fv fragments; TandAbs, Trispecific Abs (Affimed) (105-110 kDa); Darts (dual affinity retargeting; Macrogenics); Bispecific Xmabs (Xencor); Bispecific T cell engagers (Bites; Amgen; 55 kDa); Triplebodies; Tribody=Fab-scFv Fusion Protein (CreativeBiolabs) multifunctional recombinant antibody derivates (110 kDa); Duobody platform (Genmab); Dock and lock platform; Knob into hole (KIH) platform; Humanized bispecific IgG antibody (REGN1979) (Regeneron); Mab2 bispecific antibodies (F-Star); DVD-Ig-dual variable domain immunoglobulin (Abbvie); kappa-lambda bodies; tetravalent bispecific tandem Ig; and CrossMab.

In a further embodiment, the bispecific antibodies as used herein may be selected from bispecific IgG-like antibodies (BsIgG) comprising CrossMab; DAF (two-in-one); DAF (four-in-one); DutaMab; DT-IgG; Knobs-in-holes common LC; Knobs-in-holes assembly; Charge pair; Fab-arm exchange; SEEDbody; Triomab; LUZ-Y; Fcab; κλ-body; and Orthogonal Fab. These bispecific antibody formats are shown and described for example in Spiess C., Zhai Q. and Carter P. J. (2015) Molecular Immunology 67: 95-106, in particular FIG. 1 and corresponding description, e.g. p. 95-101.

In yet a further embodiment, the bispecific antibodies used herein may be selected from IgG-appended antibodies with an additional antigen-binding moiety comprising DVD-IgG; IgG(H)-scFv; scFv-(H)IgG; IgG(L)-scFv; scFV-(L)IgG; IgG(L,H)-Fv; IgG(H)-V; V(H)-IgG; IgG(L)-V; V(L)-IgG; KIH IgG-scFab; 2scFv-IgG; IgG-2scFv; scFv4-Ig; scFv4-Ig; Zybody; and DVI-IgG (four-in-one). These bispecific antibody formats are shown and described for example in Spiess C., Zhai Q. and Carter P. J. (2015) Molecular Immunology 67: 95-106, in particular FIG. 1 and corresponding description, e.g. p. 95-101.

In a yet further embodiment, the bispecific antibodies used herein may be selected from bispecific antibody fragments comprising Nanobody; Nanobody-HAS; BiTE; Diabody; DART; TandAb; scDiabody; sc-Diabody-CH3; Diabody-CH3; Triple Body; Miniantibody; Minibody; TriBi minibody; scFv-CH3 KIH; Fab-scFv; scFv-CH-CL-scFv; F(ab′)2; F(ab′)2-scFv2; scFv-KIH; Fab-scFv-Fc; Tetravalent HCAb; scDiabody-Fc; Diabody-Fc; Tandem scFv-Fc; and Intrabody. These bispecific antibody formats are shown and described for example in Spiess C., Zhai Q. and Carter P. J. (2015) Molecular Immunology 67: 95-106, in particular FIG. 1 and corresponding description, e.g. p. 95-101.

In a yet further embodiment, the bispecific antibodies used herein may be selected from bispecific fusion proteins comprising Dock and Lock; ImmTAC; HSAbody; scDiabody-HAS; and Tandem scFv-Toxin. These bispecific antibody formats are shown and described for example in Spiess C., Zhai Q. and Carter P. J. (2015) Molecular Immunology 67: 95-106, in particular FIG. 1 and corresponding description, e.g. p. 95-101.

In a yet further embodiment, the bispecific antibodies used herein may be selected from bispecific antibody conjugates comprising IgG-IgG; Cov-X-Body; and scFv1-PEG-scFv2. These bispecific antibody formats are shown and described for example in Spiess C., Zhai Q. and Carter P. J. (2015) Molecular Immunology 67: 95-106, in particular FIG. 1 and corresponding description, e.g. p. 95-101.

In yet further embodiment, the bispecific antibodies used herein may be based on any immunoglobulin class (e.g., IgA, IgG, IgM etc.) and subclass (e.g. IgA1, IgA2, IgG1, IgG2, IgG3, IgG4 etc.). In aspect, the bispecific antibodies used herein may have an IgG-like format (based on IgG, also referred to as “IgG type”), which usually comprises two heavy chains and two light chains. Examples of antibodies having an IgG-like format include a quadroma and various IgG-scFv formats (cf: Byrne H. et al. (2013) Trends Biotech, 31 (11): 621-632; FIGS. 2A-2E), whereby a quadroma is preferred, which is preferably generated by fusion of two different hybridomas. Within the IgG class, the bispecific antibodies may be based on the IgG1, IgG2, IgG3 or IgG4 subclass.

In yet a further embodiment, the bispecific antibodies used herein are in IgG-like antibody formats, which comprise for example hybrid hybridoma (quadroma), knobs-into-holes with common light chain, various IgG-scFv formats, various scFv-IgG formats, two-in-one IgG, dual V domain IgG, IgG-V, and V-IgG, which are shown for example in FIG. 3c of Chan, A. C. and Carter, P. J. (2010) Nat Rev Immu 10: 301-316 and described in said article. Further exemplary bispecific IgG-like antibody formats include for example DAF, CrossMab, IgG-dsscFv, DVD, IgG-dsFV, IgG-scFab, scFab-dsscFv and Fv2-Fc, which are shown in FIG. 1A of Weidle U. H. et al. (2013) Cancer Genomics and Proteomics 10: 1-18 and described in said article. Yet further exemplary bispecific IgG-like antibody formats include DAF (two-in-one); DAF (four-in-one); DutaMab; DT-IgG; Knobs-in-holes assembly; Charge pair; Fab-arm exchange; SEEDbody; Triomab; LUZ-Y; Fcab; κλ-body; Orthogonal Fab; DVD-IgG; IgG(H)-scFv; scFv-(H)IgG; IgG(L)-scFv; scFV-(L)IgG; IgG(L,H)-Fv; IgG(H)-V; V(H)-IgG; IgG(L)-V; V(L)-IgG; KIH IgG-scFab; 2scFv-IgG; IgG-2scFv; scFv4-Ig; scFv4-Ig; Zybody; and DVI-IgG (four-in-one) as shown and described for example in Spiess C., Zhai Q. and Carter P. J. (2015) Molecular Immunology 67: 95-106, in particular FIG. 1 and corresponding description, e.g. p. 95-101.

Bispecific antibodies, for example, can be produced by three different methods: (i) chemical conjugation, which involves chemical cross-linking; (ii) fusion of two different hybridoma cell lines; or (iii) genetic approaches involving recombinant DNA technology. The fusion of two different hybridomas produces a hybrid-hybridoma (or “quadroma”) secreting a heterogeneous antibody population including bispecific molecules. Alternative approaches included chemical conjugation of two different mAbs and/or smaller antibody fragments. Oxidative reassociation strategies to link two different antibodies or antibody fragments were found to be inefficient due to the presence of side reactions during reoxidation of the multiple native disulfide bonds. Current methods for chemical conjugation focus on the use of homo- or hetero-bifunctional crosslinking reagents. Recombinant DNA technology has yielded the greatest range of bispecific antibodies, through artificial manipulation of genes and represents the most diverse approach for bispecific antibody generation (45 formats in the past two decades; cf. Byrne H. et al. (2013) Trends Biotech, 31 (11): 621-632).

In particular by use of such recombinant DNA technology, also a variety of further multispecific antibodies have emerged recently. The term “multispecific antibodies” refers to proteins having more than one paratope and the ability to bind to two or more different epitopes. Thus, the term “multispecific antibodies” comprises bispecific antibodies as defined above, but typically also protein, e.g. antibody, scaffolds, which bind in particular to three or more different epitopes, i.e. antibodies with three or more paratopes. Such multispecific proteins, in particular with three or more paratopes, are typically achieved by recombinant DNA techniques. In the context of the present invention, the antibody may in particular also have more than two specificities, and, thus, more than two paratopes, as at least two paratopes are required according to the present invention, for example one for the target cell and the other for a T cell. Accordingly, the antibody to be used according to the invention may have further paratopes, in particular relating to further specificities, in addition to the two paratopes. Thus, the present invention also comprises multispecific antibodies. It is thus understood that the invention is not limited to bispecific antibodies, although it is referred herein in particular to bispecific antibodies, which represent the minimum requirements. What is said herein about bispecific antibodies may therefore also apply to multispecific antibodies.

The bispecific antibodies, and multispecific antibodies as defined above, are able to redirect effector cells against target cells that play key roles in disease processes. In particular, the T cell redirection bispecific antibodies used herein can, for example, bind to T cell receptor (TCR) complexes and “redirect” T cells to target cells, such as for example tumor cells. To this end, such bispecific antibodies used herein typically has at least one specificity, e.g. at least one paratope, for recruiting T cells, which is specific for T cells, preferably for T cell surface antigens, e.g. CD3, and at least one other specificity, e.g. at least one paratope, for directing T cells to tumor cells, which is specific for tumor cells, preferably a TAA on tumor cells. Such a “redirection” of a T cell to a tumor cell by a T cell redirection bispecific antibody typically results in T-cell mediated cell killing of the tumor cell.

In one embodiment, the T cell redirection therapeutic used herein comprise a first binding region with specificity against a T cell surface antigen and a second binding region with specificity against a TAA on a tumor cell.

In a further embodiment, the T cell surface antigen may be selected from CD3, CD2, CD4, CD5, CD6, CD8, CD28, CD40L, CD44, KI2L4, NKG2E, NKG2D, NKG2F, BTNL3, CD186, BTNL8, PD-1, CD195, and NKG2C. Or, the T cell surface antigen is CD3.

In a yet further embodiment, the TAA may be selected from B-cell maturation antigen (BCMA), CD123, GPRC5D, CD33, CD19, PSMA, TMEFF2, CD20, CD10, CD21, CD22, CD25, CD30, CD34, CD37, CD44v6, CD45, CD52, CD133, ROR1, B7-H6, B7-H3, HM1.24, SLAMF7, Fms-like tyrosine kinase 3 (FLT-3, CD135), chondroitin sulfate proteoglycan 4 (CSPG4, melanoma-associated chondroitin sulfate proteoglycan), epidermal growth factor receptor (EGFR), Her2, Her3, IGFR, IL3R, fibroblast activating protein (FAP), CDCP1, Derlin1, Tenascin, frizzled 1-10, VEGFR2 (KDR/FLK1), VEGFR3 (FLT4, CD309), PDGFR-alpha (CD140a), PDGFR-beta (CD140b), endoglin, CLEC14, Tem1-8, or Tie2. Further exemplary TAA on the tumor cell include A33, CAMPATH-1 (CDw52), Carcinoembryonic antigen (CEA), Carboanhydrase IX (MN/CA IX), de2-7, EGFRVIII, EpCAM, Ep-CAM, folate-binding protein, G250, c-Kit (CD117), CSF1R (CD115), HLA-DR, IGFR, IL-2 receptor, IL3R, MCSP (melanoma-associated cell surface chondroitin sulphate proteoglycane), Muc-1, prostate stem cell antigen (PSCA), prostate specific antigen (PSA), hK2, TAG-72 or a tumor cell neoantigen. Or, the TAA may be selected from BCMA, CD123, GPRC5D, CD33, CD19, PSMA, TMEFF2, CD20, CD22, CD25, CD52, ROR1, HM1.24, CD38 and SLAMF7. Or, the TAA may be selected from BCMA or CD123. Or, the TAA may be CD123.

In one embodiment, the T cell redirection therapeutic is a CD123×CD3 bispecific antibody that immunospecifically binds to CD123⁺ AML cells and CD3 T cells. The CD123×CD3 bispecific antibody may be a bispecific DuoBody® antibody as those disclosed in U.S. Pat. No. 9,850,310, which is incorporated herein by reference in its entirety. In one embodiment, the CD123×CD3 antibody comprises a HC1 having the amino acid sequence of SEQ ID NO: 1, a LC1 having the amino acid sequence of SEQ ID NO: 2, a HC2 having the amino acid sequence of SEQ ID NO: 3, and a LC2 having the amino acid sequence of SEQ ID NO: 4, wherein HC1 and LC1 pair to form a first antigen-binding site that immunospecifically binds CD123, and HC2 and LC2 pair to form a second antigen-binding site that immunospecifically binds CD3.

SEQ ID NO: 1
EVQLVQSGAEVKKPGESLKISCKGSGYSFTSYWISWVRQMPGKGLEWMGI
IDPSDSDTRYSPSFQGQVTISADKSISTAYLOWSSLKASDTAMYYCARGD
GSTDLDYWGQGTLVTVSSASTKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEAAGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGK
SEQ ID NO: 2
EIVLTQSPGTLSLSPGERATLSCRASQSVSSSYLAWYQQKPGQAPRLLIY
GASSRATGIPDRFSGSGSGTDFTLTISRLEPEDFAVYYCQQDYGFPWTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
SEQ ID NO: 3
EVQLVESGGGLVQPGGSLKLSCAASGFTFNTYAMNWVRQASGKGLEWVGR
IRSKYNAYATYYAASVKGRFTISRDDSKNTAYLQMNSLKTEDTAVYYCTR
HGNFGNSYVSWFAYWGQGTLVTVSSASTKGPSVFPLAPCSRSTSESTAAL
GCLVKDYFPEPVTVSWNSGALTSGVHTFPAVLQSSGLYSLSSVVTVPSSS
LGTKTYTCNVDHKPSNTKVDKRVESKYGPPCPPCPAPEAAGGPSVFLFPP
KPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTP
PVLDSDGSFLLYSKLTVDKSRWQEGNVFSCSVMHEALHNHYTQKSLSLSL
GK
SEQ ID NO: 4
QAVVTQEPSLTVSPGGTVTLTCRSSTGAVTTSNYANWVQQKPGQAPRGLI
GGTNKRAPGTPARFSGSLLGGKAALTLSGAQPEDEAEYYCALWYSNLWVF
GGGTKLTVLGQPKAAPSVTLFPPSSEELQANKATLVCLISDFYPGAVTVA
WKADSSPVKAGVETTTPSKQSNNKYAASSYLSLTPEQWKSHRSYSCQVTH
EGSTVEKTVAPTECS

In one embodiment, the T cell redirection therapeutic is a BCMA×CD3 bispecific antibody that immunospecifically binds to BCMA⁺ MM cells and CD3 T cells. The BCMA×CD3 bispecific antibodies may be selected from those disclosed in WO2007117600, WO2009132058, WO2012066058, WO2012143498, WO2013072406, WO2013072415, WO2014122144, and U.S. Pat. No. 10,072,088, which are incorporated herein by reference in their entirety.

In one embodiment, the T cell redirection therapeutic is a BCMA×CD3 bispecific antibody that comprises a first heavy chain (HC1) having the amino acid sequence of SEQ ID NO: 5, a first light chain (LC1) having the amino acid sequence of SEQ ID NO: 6, a second heavy chain (HC2) having the amino acid sequence of SEQ ID NO: 7, and a second light chain (LC2) having the amino acid sequence of SEQ ID NO: 8, wherein HC1 and LC1 pair to form a first antigen-binding site that immunospecifically binds BCMA, and HC2 and LC2 pair to form a second antigen-binding site that immunospecifically binds CD3.

SEQ ID NO: 5
QLQLQESGPGLVKPSETLSLTCTVSGGSISSGSYFWGWIRQPPGKGLEWI
GSIYYSGITYYNPSLKSRVTISVDTSKNQFSLKLSSVTAADTAVYYCARH
DGAVAGLFDYWGQGTLVTVSSASTKGPSVFPLAPCSRSTSESTAALGCLV
KDYFPEPVTVSWNSGALTSGVHTFPAVLQSSGLYSLSSVVTVPSSSLGTK
TYTCNVDHKPSNTKVDKRVESKYGPPCPPCPAPEAAGGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK
SEQ ID NO: 6
SYVLTQPPSVSVAPGQTARITCGGNNIGSKSVHWYQQPPGQAPVVVVYDD
SDRPSGIPERFSGSNSGNTATLTISRVEAGDEAVYYCQVWDSSSDHVVFG
GGTKLTVLGQPKAAPSVTLFPPSSEELQANKATLVCLISDFYPGAVTVAW
KGDSSPVKAGVETTTPSKQSNNKYAASSYLSLTPEQWKSHRSYSCQVTHE
GSTVEKTVAPTECS
SEQ ID NO: 7
EVQLVESGGGLVQPGGSLRLSCAASGFTFNTYAMNWVRQAPGKGLEWVAR
IRSKYNNYATYYAASVKGRFTISRDDSKNSLYLQMNSLKTEDTAVYYCAR
HGNFGNSYVSWFAYWGQGTLVTVSSASTKGPSVFPLAPCSRSTSESTAAL
GCLVKDYFPEPVTVSWNSGALTSGVHTFPAVLQSSGLYSLSSVVTVPSSS
LGTKTYTCNVDHKPSNTKVDKRVESKYGPPCPPCPAPEAAGGPSVFLFPP
KPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTP
PVLDSDGSFLLYSKLTVDKSRWQEGNVFSCSVMHEALHNHYTQKSLSLSL
GK
SEQ ID NO: 8
QTVVTQEPSLTVSPGGTVTLTCRSSTGAVTTSNYANWVQQKPGQAPRGLI
GGTNKRAPGTPARFSGSLLGGKAALTLSGVQPEDEAEYYCALWYSNLWVF
GGGTKLTVLGQPKAAPSVTLFPPSSEELQANKATLVCLISDFYPGAVTVA
WKADSSPVKAGVETTTPSKQSNNKYAASSYLSLTPEQWKSHRSYSCQVTH
EGSTVEKTVAPTECS

### Anti-CD44 Therapeutics

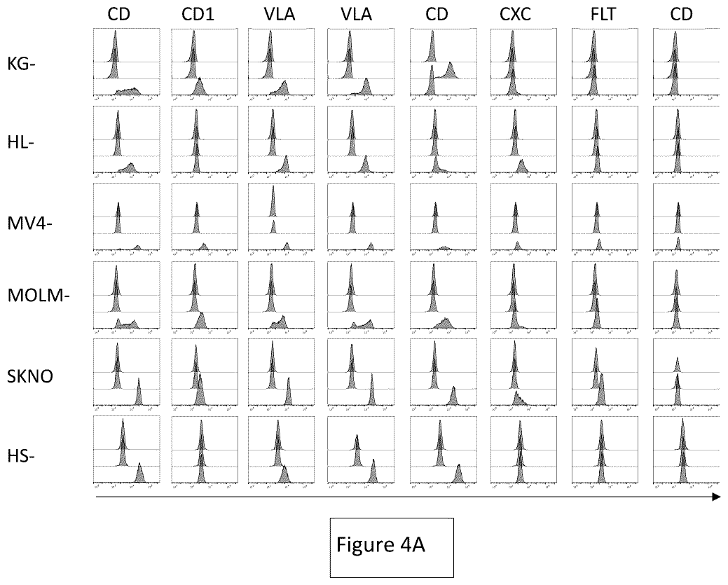

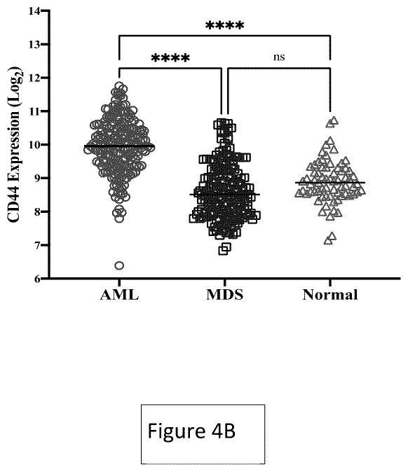

The CD44 antigen is a cell-surface glycoprotein involved in cell-cell interactions, cell adhesion and migration. In humans, the CD44 antigen is encoded by the CD44 gene on Chromosome 11. CD44 has been shown to contribute to the adherent properties of hematopoietic stem cells (HSCs) and leukemic stem cells (LSCs) (see e.g., Schroeder et al., “mesenchymal Stromal Cells in Myeloid Malignancies”, Blood Research Vol 51, No. 4, December 2016 and Pleyer et al., “Mesenchymal Stem and Progenitor Cells in Normal and Dysplastic Hematopoiesis—Masters of Survival and Clonality”, Int. J. Mol. Sci. 2016, 17, 1009). In AML, CD44 is upregulated on AML blasts and LSCs and functions as a survival signal through a variety of pathways (see e.g., Gul-Uludag et al, “Polymeric nanoparticle-mediated silencing of CD44 receptor in CD34+ acute myeloid leukemia cells”, Leukemia research. 2014; 38(11): 1299-1308; Garg et al., “Antigen expression on a putative leukemic stem cell population and AML blast”, Int J Hematol. 2016; 103(5):567-571; Chen et al., “Bone marrow stromal cells protect acute myeloid leukemia cells from anti-CD44 therapy partly through regulating PI3K/Akt-p27(Kip1) axis”, Mol Carcinog. 2015; 54(12):1678-1685; and Gadhoum et al., “Anti-CD44 antibodies inhibit both mTORC1 and mTORC2: a new rationale supporting CD44-induced AML differentiation therapy”, Leukemia. 2016; 30(12):2397-2401). And as provided herebelow in the Example section, the CD44 protein is consistently expression in various AML cell lines (FIG. 4A). CD44 mRNA expression in primary AML cells (n=202) also was assessed and a significant upregulation was observed compared to myelodysplastic syndrome (MDS; n=164) and normal control (n=69; FIG. 4B). This is consistent with the idea that CD44 expression may be important in disease progression from MDS to AML disease.

Any suitable anti-CD44 therapeutics maybe used herein, which include, disclosed, without limitation, anti-CD44 antibodies or CD44-binding fragments derived therefrom, CD44 antagonists, etc.

In one embodiment, the anti-CD44 therapeutic used herein may be an anti-CD44 antibody or a CD44-binding fragment derived therefrom, such as Fab, Fab′, F(ab′)2, and F(v) fragments; heavy chain monomers or dimers; light chain monomers or dimers; and dimers consisting of one heavy chain and one light chain are also contemplated herein. Such antibody fragments may be produced by chemical methods, e.g., by cleaving an intact antibody with a protease, such as pepsin or papain, or via recombinant DNA techniques, e.g., by using host cells transformed with truncated heavy and/or light chain genes. Heavy and light chain monomers may similarly be produced by treating an intact antibody with a reducing agent such as dithiothreitol or ß-mercaptoethanol or by using host cells transformed with DNA encoding either the desired heavy chain or light chain or both, or, such as, a monoclonal antibody or an antibody fragment thereof.

Any suitable anti-CD44 antibodies or CD44-binding fragments capable of blocking the CD44 functionality may be used herein, which include, without limitation, RG7356, anti-CD44 ab clone A3D8, anti-CD44 ab clone H90, anti-CD44 ab clone 515. Suitable anti-CD44 antibodies also may be selected from the those disclosed in U.S. Pat. No. 7,361,347, US20140308301, US20070237761, US20090004103, US20100092484, and US20050214283.

### VLA-4 Adhesion Pathway Inhibitor

Very late antigen-4 (VLA-4), also known as called α4β1, is a member of the β1 integrin family of cell surface receptors. VLA-4 contains a α4 chain and a β1 chain and is involved in cell-cell interactions. Its expression is mainly restricted to lymphoid and myeloid cells. It is a key player in cell adhesion. Studies also have shown that VLA-4 plays an important role in mediating AML/MM-stroma interactions in BM. Vascular cell adhesion molecule-1 (VCAM-1) (expressed by osteoblasts and endothelial cells) and fibronectin (a component of the extracellular matrix) are two ligands for VLA-4.

The VLA-4 adhesion pathway inhibitors used herein may be any molecule that is capable of blocking the VLA-4 mediated adhesion pathway.

For example, the VLA-4 adhesion pathway inhibitor used herein may be an anti-VLA-4 antibody or a VLA-4-binding fragment derived therefrom, such as Fab, Fab′, F(ab′)2, and F(v) fragments; heavy chain monomers or dimers; light chain monomers or dimers; and dimers consisting of one heavy chain and one light chain are also contemplated herein. Such antibody fragments may be produced by chemical methods, e.g., by cleaving an intact antibody with a protease, such as pepsin or papain, or via recombinant DNA techniques, e.g., by using host cells transformed with truncated heavy and/or light chain genes. Heavy and light chain monomers may similarly be produced by treating an intact antibody with a reducing agent such as dithiothreitol or β-mercaptoethanol or by using host cells transformed with DNA encoding either the desired heavy chain or light chain or both, or, such as, a monoclonal antibody or an antibody fragment thereof.

Any suitable anti-VLA-4 antibodies or VLA-4-binding fragments capable of blocking the VLA-4-mediated adhesion pathway may be used herein, which include, without limitation, natalizumab and those disclosed in U.S. Pat. No. 6,602,503 and U.S. Patent Application Publication No. US20140161794 A1, which are incorporated herein by reference in their entirety.

In certain embodiments, the VLA-4 adhesion pathway inhibitors used herein may be VLA-4 antagonists that are capable of blocking the VLA-4-mediated adhesion pathway. Exemplary VLA-4 antagonists used herein include, without limitation, VLA-4 antagonists from Tocris Bioscience (e.g., BIO1211, TCS2314, BIO5192, and TR14035).

Inasmuch as VCAM-1 and fibronectin are ligands of VLA-4, the VLA-4 adhesion pathway inhibitors also may include antagonists (including antibodies) of VCAM-1 or fibronectin.

### Pharmaceutical Compositions

Further disclosed herein are pharmaceutical compositions comprising a T cell redirection therapeutic, as disclosed above, and an anti-CD44 therapeutic, as disclosed above, and a pharmaceutically acceptable carrier. Polynucleotides, polypeptides, host cells, and/or engineered immune cells of the invention and compositions comprising them are also useful in the manufacture of a medicament for therapeutic applications mentioned herein. In certain embodiments, the pharmaceutical compositions are separate compositions comprising a T cell redirection therapeutic, as disclosed above, and an anti-CD44 therapeutic, as disclosed above, and a pharmaceutically acceptable carrier. In other embodiments, the pharmaceutical compositions are not separate compositions comprising a T cell redirection therapeutic, as disclosed above, and an anti-CD44 therapeutic, as disclosed above, and a pharmaceutically acceptable carrier.

In one embodiment, the pharmaceutical composition disclosed herein may further comprise a VLA-4 adhesion pathway inhibitor, as disclosed above. In one aspect, the pharmaceutical compositions are separate compositions comprising a T cell redirection therapeutic, an anti-CD44 therapeutic, and a VLA-4 adhesion pathway inhibitor. In another aspect, the pharmaceutical compositions are separate compositions comprising a first composition comprising a T cell redirection therapeutic and a second composition comprising an anti-CD44 therapeutic and a VLA-4 adhesion pathway inhibitor. In yet another aspect, the pharmaceutical compositions are not separate compositions and the pharmaceutical compositions comprise a T cell redirection therapeutic, an anti-CD44 therapeutic, a VLA-4 adhesion pathway inhibitor, and a pharmaceutically acceptable carrier.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in a polynucleotide, polypeptide, host cell, and/or engineered immune cell pharmaceutical composition can be used in the invention.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carrier may be used in formulating the pharmaceutical compositions of the invention.

In one embodiment of the disclosure, the pharmaceutical composition is a liquid formulation. A preferred example of a liquid formulation is an aqueous formulation, i.e., a formulation comprising water. The liquid formulation can comprise a solution, a suspension, an emulsion, a microemulsion, a gel, and the like.

In one embodiment, the pharmaceutical composition can be formulated as an injectable which can be injected, for example, via an injection device (e.g., a syringe or an infusion pump). The injection can be delivered subcutaneously, intramuscularly, intraperitoneally, intravitreally, or intravenously, for example.

In another embodiment, the pharmaceutical composition is a solid formulation, e.g., a freeze-dried or spray-dried composition, which can be used as is, or whereto the physician or the patient adds solvents, and/or diluents prior to use.

### Methods of Use

In another general aspect, the invention relates to a method of treating a cancer in a subject in need thereof, comprising administering to the subject pharmaceutical compositions comprising the T cell redirection therapeutic, the anti-CD44 therapeutic as disclosed herein. In one embodiment, the pharmaceutical composition may further comprise the VLA-4 adhesion pathway inhibitor, as disclosed herein.

In another general aspect, the invention relates to a method of killing cancer cells comprising subjecting the cancer cells to compositions comprising the T cell redirection therapeutic and the anti-CD44 therapeutic, as disclosed herein. In one embodiment, the pharmaceutical composition may further comprise the VLA-4 adhesion pathway inhibitor, as disclosed herein.

The subject may have a newly diagnosed cancer or is relapsed or refractory to a prior anti-cancer therapy. The cancer may be a hematological malignancy or a solid tumor.

According to embodiments of the invention, the pharmaceutical compositions comprise a therapeutically effective amount of the T cell redirection therapeutic, the anti-CD44 therapeutic, and the optional VLA-4 adhesion pathway inhibitor, as disclosed herein. As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

As used herein with reference to the T cell redirection therapeutic and the anti-CD44 therapeutic, a therapeutically effective amount means an amount of the T cell redirection therapeutic in combination with the anti-CD44 therapeutic that modulates an immune response in a subject in need thereof. Also, as used herein with reference to the T cell redirection therapeutic, a therapeutically effective amount means an amount of the T cell redirection therapeutic with the anti-CD44 therapeutic that results in treatment of a disease, disorder, or condition; prevents or slows the progression of the disease, disorder, or condition; or reduces or completely alleviates symptoms associated with the disease, disorder, or condition.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

According to particular embodiments, provided are pharmaceutical compositions used in the treatment of a cancer. For cancer therapy, the provided pharmaceutical compositions can be used in combination with another treatment including, but not limited to, a chemotherapy, an anti-CD20 mAb, an anti-TIM-3 mAb, an anti-LAG-3 mAb, an anti-EGFR mAb, an anti-HER-2 mAb, an anti-CD19 mAb, an anti-CD33 mAb, an anti-CD47 mAb, an anti-CD73 mAb, an anti-DLL-3 mAb, an anti-apelin mAb, an anti-TIP-1 mAb, an anti-FOLR1 mAb, an anti-CTLA-4 mAb, an anti-PD-L1 mAb, an anti-PD-1 mAb, other immuno-oncology drugs, an antiangiogenic agent, a radiation therapy, an antibody-drug conjugate (ADC), a targeted therapy, or other anticancer drugs.

According to particular embodiments, the methods of treating cancer in a subject in need thereof comprise administering to the subject T cell redirection therapeutic in combination with an anti-CD44 therapeutic and an optional VLA-4 adhesion pathway inhibitor as disclosed herein.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., the T cell redirection therapeutic described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., the VLA-4 adhesion pathway inhibitor) to a subject.

### Kits

In another general aspect, provided herein are kits, unit dosages, and articles of manufacture comprising the T cell redirection therapeutic as disclosed herein, the anti-CD44 therapeutic as disclosed herein, optionally the VLA-4 adhesion pathway inhibitor as disclosed herein, and optionally a pharmaceutical carrier. In certain embodiments, the kit preferably provides instructions for its use.

In another particular aspect, provided herein are kits comprising (1) a T cell redirection therapeutic as disclosed herein and (2) an anti-CD44 therapeutic as disclosed herein.

In another particular aspect, provided herein are kits comprising (1) a T cell redirection therapeutic as disclosed herein, (2) an anti-CD44 therapeutic as disclosed herein, and (3) a VLA-4 adhesion pathway inhibitor as disclosed herein.

In another particular aspect, provided herein are kits comprising pharmaceutical compositions comprising (1) a T cell redirection therapeutic as disclosed herein, (2) an anti-CD44 therapeutic as disclosed herein, and (3) a pharmaceutically acceptable carrier.

In another particular aspect, provided herein are kits comprising pharmaceutical compositions comprising (1) a T cell redirection therapeutic as disclosed herein, (2) an anti-CD44 therapeutic as disclosed herein, (3) a VLA-4 adhesion pathway inhibitor as disclosed herein, and (4) a pharmaceutically acceptable carrier.

### Embodiments

Embodiment 1 of the invention includes the pharmaceutical compositions comprising a T cell redirection therapeutic and an anti-CD44 therapeutic, wherein, the T cell redirection therapeutic comprises a first binding region that immunospecifically binds a T cell surface antigen and a second binding region that immunospecifically binds a tumor associated antigen (TAA).
Embodiment 2 of the invention includes the pharmaceutical compositions of embodiment 1, wherein the compositions further comprise a pharmaceutically acceptable carrier.
Embodiment 3 of the invention includes the pharmaceutical compositions of embodiment 1 or 2, wherein the T cell redirection therapeutic is an antibody or antigen-binding fragment thereof.
Embodiment 4 of the invention includes the pharmaceutical compositions of any one of embodiments 1-3, wherein the T cell surface antigen is selected from the group consisting of CD3, CD2, CD4, CD5, CD6, CD8, CD28, CD40L, CD44, KI2L4, NKG2E, NKG2D, NKG2F, BTNL3, CD186, BTNL8, PD-1, CD195, and NKG2C.
Embodiment 5 of the invention includes the pharmaceutical compositions of embodiment 4, wherein the T cell surface antigen is CD3.
Embodiment 6 of the invention includes the pharmaceutical compositions of any one of embodiments 1-5, wherein the TAA is selected from the group consisting of CD123, BCMA, GPRC5D, CD33, CD19, PSMA, TMEFF2, CD20, CD22, CD25, CD52, ROR1, HM1.24, CD38, and SLAMF7.
Embodiment 7 of the invention includes the pharmaceutical compositions of embodiment 6, wherein the T cell redirection therapeutic is a CD123×CD3 bispecific antibody having a first antigen-binding site that immunospecifically binds CD123 and a second antigen-binding site that immunospecifically binds CD3.
Embodiment 8 of the invention includes the pharmaceutical compositions of embodiment 7, wherein the CD123×CD3 bispecific antibody comprises a first heavy chain (HC1), a first light chain (LC1), a second heavy chain (HC2), and a second light chain (LC2), and wherein the HC1 and the LC1 pair to form the first antigen-binding site and the HC2 and the LC2 pair to form the second antigen-binding site.
Embodiment 9 of the invention includes the pharmaceutical compositions of embodiment 8, wherein the HC1 comprises the amino acid sequence of SEQ ID NO: 1, the LC1 comprises the amino acid sequence of SEQ ID NO: 2, the HC2 comprises the amino acid sequence of SEQ ID NO: 3, and the LC2 comprises the amino acid sequence of SEQ ID NO: 4.
Embodiment 10 of the invention includes the pharmaceutical compositions of embodiment 6, wherein the T cell redirection therapeutic is a BCMA×CD3 bispecific antibody having a first antigen-binding site that immunospecifically binds CD123 and a second antigen-binding site that immunospecifically binds CD3.
Embodiment 11 of the invention includes the pharmaceutical compositions of embodiment 10, wherein the BCMA×CD3 bispecific antibody comprises a first heavy chain (HC1), a first light chain (LC1), a second heavy chain (HC2), and a second light chain (LC2), and wherein the HC1 and the LC1 pair to form the first antigen-binding site and the HC2 and the LC2 pair to form the second antigen-binding site.
Embodiment 12 of the invention includes the pharmaceutical compositions of embodiment 11, wherein the HC1 comprises the amino acid sequence of SEQ ID NO: 5, the LC1 comprises the amino acid sequence of SEQ ID NO: 6, the HC2 comprises the amino acid sequence of SEQ ID NO: 7, and the LC2 comprises the amino acid sequence of SEQ ID NO: 8.
Embodiment 13 of the invention includes the pharmaceutical compositions of any one of embodiments 1-12, wherein the anti-CD44 therapeutic is an anti-CD44 antibody or antigen-binding fragment thereof.
Embodiment 14 of the invention includes the pharmaceutical compositions of embodiment 13, wherein the anti-CD44 antibody or antigen-binding fragment thereof is selected from the group consisting of monoclonal antibodies, scFv, Fab, Fab′, F(ab′)2, and F(v) fragments, heavy chain monomers or dimers, light chain monomers or dimers, and dimers consisting of one heavy chain and one light chain.
Embodiment 15 of the invention includes the pharmaceutical compositions of any one of embodiments 1-12, wherein the anti-CD44 therapeutic is a CD44 antagonist.
Embodiment 16 of the invention includes the pharmaceutical compositions of any one of embodiments 1-16, wherein the composition further comprises a VLA-4 adhesion pathway inhibitor.
Embodiment 17 of the invention includes the pharmaceutical composition of embodiment 16, wherein the VLA-4 adhesion pathway inhibitor is an anti-VLA-4 antibody or antigen-binding fragment thereof.
Embodiment 18 of the invention includes the pharmaceutical compositions of embodiment 17, wherein the anti-VLA-4 antibody or antigen-binding fragment thereof is selected from the group consisting of monoclonal antibodies, scFv, Fab, Fab′, F(ab′)2, and F(v) fragments, heavy chain monomers or dimers, light chain monomers or dimers, and dimers consisting of one heavy chain and one light chain.
Embodiment 19 of the invention includes the pharmaceutical composition of embodiment 16, wherein the VLA-4 adhesion pathway inhibitor is a VLA-4 antagonist.
Embodiment 20 of the invention includes the pharmaceutical compositions of embodiment 19, wherein the VLA-4 antagonist is selected from the group consisting of BIO1211, TCS2314, BIO5192, and TR14035.
Embodiment 21 of the invention includes methods of killing cancer cells, comprising subjecting cancer cells to therapeutically effective amounts of the pharmaceutical composition of any one of embodiments 1-20 wherein the cancer cells undergo some form of cell death.
Embodiment 22 of the invention includes methods of embodiment 21, wherein the T cell redirection therapeutic and the anti-CD44 therapeutic are administered simultaneously or sequentially.
Embodiment 23 of the invention includes methods of embodiment 22, wherein the anti-CD44 therapeutic is administered prior to administration of the T cell redirection therapeutic.
Embodiment 24 of the invention includes methods of embodiment 22, wherein the anti-CD44 therapeutic is administered after administration of the T cell redirection therapeutic.
Embodiment 25 of the invention includes methods of killing cancer cells comprising disrupting cell-cell contact between cancer cells and stromal cells, comprising subjecting cancer cells to therapeutically effective amounts of the pharmaceutical composition of any one of embodiments 1-20 wherein the cancer cells undergo some form of cell death.
Embodiment 26 of the invention includes methods of embodiment 25, wherein the T cell redirection therapeutic and the anti-CD44 therapeutic are administered simultaneously or sequentially.
Embodiment 27 of the invention includes methods of embodiment 26, wherein the anti-CD44 therapeutic is administered prior to administration of the T cell redirection therapeutic.
Embodiment 28 of the invention includes methods of embodiment 26, wherein the anti-CD44 therapeutic is administered after administration of the T cell redirection therapeutic.
Embodiment 29 of the invention includes methods of killing cancer cells comprising increasing T cell-dependent cytotoxicity, comprising subjecting cancer cells to therapeutically effective amounts of the pharmaceutical composition of any one of embodiments 1-20 wherein cancer cells undergo some form of cell death.
Embodiment 30 of the invention includes methods of embodiment 29, wherein the T cell redirection therapeutic and the anti-CD44 therapeutic are administered simultaneously or sequentially.
Embodiment 31 of the invention includes methods of embodiment 30, wherein the anti-CD44 therapeutic is administered prior to administration of the T cell redirection therapeutic.
Embodiment 32 of the invention includes methods of embodiment 30, wherein the anti-CD44 therapeutic is administered after administration of the T cell redirection therapeutic.
Embodiment 33 of the invention includes methods of killing cancer cells comprising disrupting cell-cell contact between cancer cells and stromal cells and increasing T cell-dependent cytotoxicity, comprising subjecting cancer cells to therapeutically effective amounts of the pharmaceutical composition of any one of embodiments 1-20 wherein the cancer cells undergo some form of cell death.
Embodiment 34 of the invention includes methods of embodiment 33, wherein the T cell redirection therapeutic and the anti-CD44 therapeutic are administered simultaneously or sequentially.
Embodiment 35 of the invention includes methods of embodiment 34, wherein the anti-CD44 therapeutic is administered prior to administration of the T cell redirection therapeutic.
Embodiment 36 of the invention includes methods of embodiment 34, wherein the anti-CD44 therapeutic is administered after administration of the T cell redirection therapeutic.
Embodiment 37 of the invention includes methods of altering immunosuppression in a tumor microenvironment, comprising subjecting a tumor microenvironment to therapeutically effective amounts of the pharmaceutical composition of any one of embodiments 1-20 wherein the immunosuppression is lessened in the tumor microenvironment and cancer cells undergo some form of cell death.
Embodiment 38 of the invention includes methods of embodiment 37, wherein the T cell redirection therapeutic and the anti-CD44 therapeutic are administered simultaneously or sequentially.
Embodiment 39 of the invention includes methods of embodiment 38, wherein the anti-CD44 therapeutic is administered prior to administration of the T cell redirection therapeutic.
Embodiment 40 of the invention includes methods of embodiment 38, wherein the anti-CD44 therapeutic is administered after administration of the T cell redirection therapeutic.
Embodiment 41 of the invention includes methods of altering immunosuppression in a tumor microenvironment comprising disrupting cell-cell contact between cancer cells and stromal cells, comprising subjecting the tumor microenvironment to therapeutically effective amounts of the pharmaceutical composition of any one of embodiments 1-20 wherein immunosuppression is lessened in the tumor microenvironment can cancer cells undergo some form of cell death.
Embodiment 42 of the invention includes methods of embodiment 41, wherein the T cell redirection therapeutic and the anti-CD44 therapeutic are administered simultaneously or sequentially.
Embodiment 43 of the invention includes methods of embodiment 42, wherein the anti-CD44 therapeutic is administered prior to administration of the T cell redirection therapeutic.
Embodiment 44 of the invention includes methods of embodiment 42, wherein the anti-CD44 therapeutic is administered after administration of the T cell redirection therapeutic.
Embodiment 45 of the invention includes methods of altering immunosuppression in a tumor microenvironment comprising increasing T cell-dependent cytotoxicity, comprising subjecting a tumor microenvironment to therapeutically effective amounts of the pharmaceutical composition of any one of embodiments 1-20 wherein immunosuppression is lessened in the tumor microenvironment and cancer cells undergo some form of cell death.
Embodiment 46 of the invention includes methods of embodiment 45, wherein the T cell redirection therapeutic and the anti-CD44 therapeutic are administered simultaneously or sequentially.
Embodiment 47 of the invention includes methods of embodiment 46, wherein the anti-CD44 therapeutic is administered prior to administration of the T cell redirection therapeutic.
Embodiment 48 of the invention includes methods of embodiment 46, wherein the anti-CD44 therapeutic is administered after administration of the T cell redirection therapeutic.
Embodiment 49 of the invention includes methods of altering immunosuppression in a tumor microenvironment comprising disrupting cell-cell contact between cancer cells and stromal cells and increasing T cell-dependent cytotoxicity, comprising subjecting a tumor microenvironment to therapeutically effective amounts of the pharmaceutical composition of any of embodiments 1-20 wherein immunosuppression is lessened in the tumor microenvironment and cancer cells undergo some form of cell death.
Embodiment 50 of the invention includes methods of embodiment 49, wherein the T cell redirection therapeutic and the anti-CD44 therapeutic are administered simultaneously or sequentially.
Embodiment 51 of the invention includes methods of embodiment 50, wherein the anti-CD44 therapeutic is administered prior to administration of the T cell redirection therapeutic.
Embodiment 52 of the invention includes methods of embodiment 50, wherein the anti-CD44 therapeutic is administered after administration of the T cell redirection therapeutic.
Embodiment 53 of the invention includes methods of treating cancer in a subject in need thereof, comprising administering to the subject a therapeutically effective amount of the pharmaceutical composition of any one of embodiments 1-20.
Embodiment 54 of the invention includes methods of embodiment 53, wherein the subject has a newly diagnosed cancer.
Embodiment 55 of the invention includes methods of embodiment 53, wherein the subject is relapsed or refractory to a prior anti-cancer therapy.
Embodiment 56 of the invention includes methods of any one of embodiments 53-55, wherein the cancer is a hematological malignancy or a solid tumor.
Embodiment 57 of the invention includes methods of embodiment 57, wherein the subject has AML or MM.
Embodiment 58 of the invention includes methods of any one of embodiments 53-57, wherein the T cell redirection therapeutic and the anti-CD44 therapeutic are administered simultaneously or sequentially.
Embodiment 59 of the invention includes methods of embodiment 58, wherein the anti-CD44 therapeutic is administered prior to administration of the T cell redirection therapeutic.
Embodiment 60 of the invention includes methods of embodiment 58, wherein the anti-CD44 therapeutic is administered after administration of the T cell redirection therapeutic.
Embodiment 61 of the invention includes kits comprising the pharmaceutical composition of any one of embodiments 1-20, wherein the pharmaceutical compositions are packaged separately.
Embodiment 62 of the invention includes kits comprising the pharmaceutical compositions of any one of embodiments 1-20 wherein the pharmaceutical compositions are packaged together.
Embodiment 63 of the invention includes methods of any one of embodiments 21-60 further comprising administering a VLA-4 adhesion pathway inhibitor.
Embodiment 64 of the invention includes methods of embodiment 63, wherein the VLA-4 adhesion pathway inhibitor is administered simultaneously or sequentially in reference to therapeutically effective amounts of the pharmaceutical composition of any of embodiments 1-20.

### Examples

### Materials and Methods

### Antibody Design

Bispecific antibodies were produced targeting human CD123 and CD3, in which the anti-CD123 antibody and the anti-CD3 antibody were joined together post-purification by generating a controlled fragment antigen binding arm exchange using the Genmab technology (17, 18). This resulted in a monovalent binding, bi-functional DuoBody® antibody which specifically binds to human CD123+ AML cells and CD3 T cells. To minimize antibody-mediated effector functions, mutations were introduced in the Fc domain to reduce interactions with Fcγ receptors. The CD123×CD3 bispecific used in the following experiments comprise a first heavy chain (HC1), a second heavy chain (HC2), a first light chain (LC1), and a second light chain (LC2), in which HC1 and LC1 pair to form a first antigen-binding site that immunospecifically binds CD123, and HC2 and LC2 pair to form a second antigen-binding site that immunospecifically binds CD3. The CD123×CD3 bispecific used in the following examples comprises HC1 having the amino acid sequence of SEQ ID NO: 1, LC1 having the amino acid sequence of SEQ ID NO: 2, HC2 having the amino acid sequence of SEQ ID NO: 3, and LC2 having the amino acid sequence of SEQ ID NO: 4.

### In Vitro Cytotoxicity Assays

Tumor cell lines (KG-1, HL-60, MOLM-13, MV4-11, and SKNO-1) were counted and washed with DPBS^{-/-} (without calcium or magnesium) twice before incubating with 5-15 mL DPBS^{-/-} containing the viable dye, carboxyfluorescein succinimidyl ester (CFSE; resuspended in 150 μL dimethyl sulfoxide and diluted 1:10,000) for 8 minutes at room temperature (RT) in the dark. Staining was quenched with an equal amount heat inactivated (HI) fetal bovine serum (FBS). AML target cells were washed in complete medium twice, before resuspension at 3×10⁵ cells/mL in complete medium. The HS-5 stromal cells were harvested and washed with complete media and resuspended to 3×10⁵ cells/mL in complete medium. Frozen primary adipocytes, BM stromal, MSCs, T cells, and osteoblasts were thawed, washed once in complete media and resuspended (stromal cells: 3×10⁵ cells/mL and T cells: 6×10⁵ cells/mL) in complete medium. Equal volume (67 μL) of AML, stromal and T cells were added to a 96 well TC-treated U bottom plate [effector:target:stroma (2:1:1)]. In control wells with no stromal cells, 67 μL of complete media were added. Next, 0.5 mg/mL of sterile, azide-free, low-endotoxin human immunoglobulin (IgG) obtained from Southern Biotech (Birmingham, Alabama) was added as an Fc block. The 96 well plates were incubated overnight at 37° C. with 5% CO₂.

The CD123×CD3 bispecific antibody was diluted to a final starting concentration of 100 nM in complete medium and further diluted 10-fold and added to appropriate wells. All plates were incubated at 37° C. with 5% CO₂ for an additional 24-48 hours. The cells were then washed with DPBS and stained with various markers containing LIVE/DEAD Near-IR for 30 minutes at RT. Finally, cells were washed and resuspended in stain buffer.

Studies containing anti-CD44 antibody were obtained from R&D Systems (Minneapolis, Minnesota), reconstituted in DPBS^{-/-} and added at 20 μg/mL to conditions indicated at the time of adding the cells (24 hours prior to CD123×CD3 addition).

### Flow Cytometry Analysis

Surface expression of CD44, VLA-4, VLA-5, CXCR4, CD123, CD54, CD3, and Flt3 [antibodies were purchased from Biolegend (San Diego, California)] was evaluated on KG-1^{wt}, KG-1^{CD44 KO}, MV4-11^{wt}, MV4-11^{CD44 KO}, HL-60^{wt}, HL-60^{CD44 KO}, HS-5^{wt}, and HS-5^{CD44 KO} cell lines. The AML cell lines were grown in complete media to ˜70% confluence, harvested and washed once and resuspended to 1×10⁶ cells/mL in DPBS^{-/-}. The HS-5 stromal cell line was cultured in complete media, washed once and resuspended in DPBS^{-/-}.

All of the cell lines were added to a 96 U bottom plate at 100 μL/well (1×10⁵ cells/well). The plate was centrifuged, and the supernatant was discarded. The cells were stained individually for each antigen or isotype with the LIVE/DEAD dye in DPBS^{-/-} at RT.

### CD44 Expression of AML and MDS Samples

CD44 expression was evaluated from the Mills Heme (GSE15061) study in AML, myelodysplastic syndrome (MDS) and normal controls and plotted using GraphPad Prism version 9 (San Diego, California).

### Statistical Methods

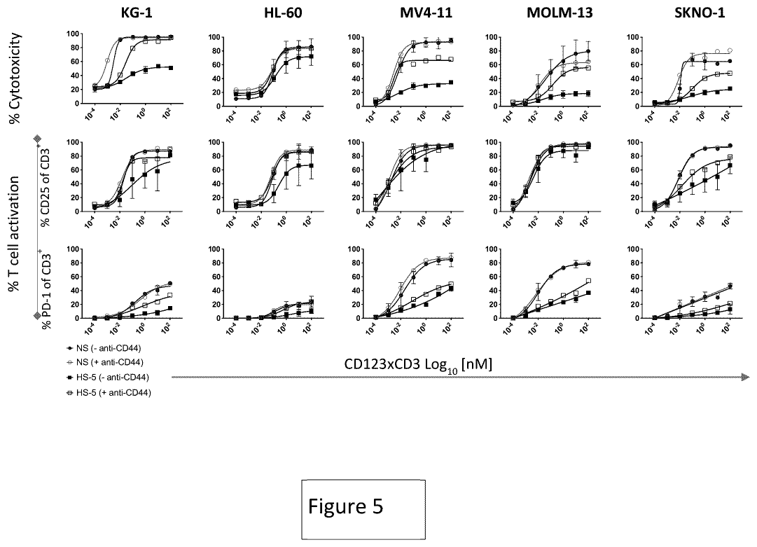

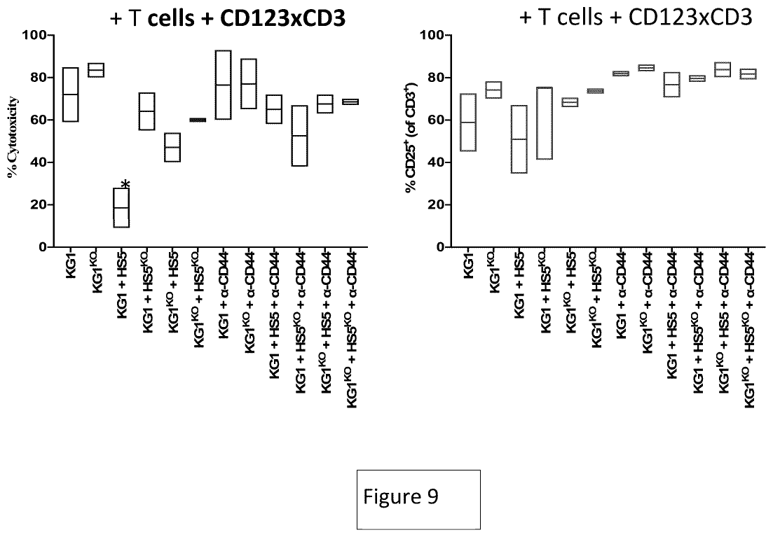

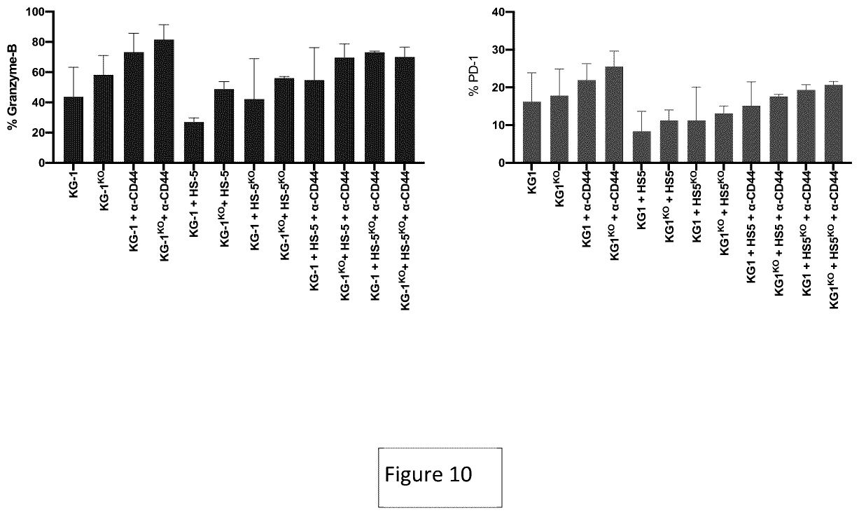

All data were analyzed by GraphPad software Prism version 9. Non-linear regression (four parameter) curve fit was utilized for FIGS. 1 and 5. The EC₅₀ and max cytotoxicity values were derived from the aforementioned analysis. An F test analysis was performed on the 4-parameter nonlinear fit by comparing the top parameter (alpha=0.05) to evaluate statistical significance for FIG. 1. ANOVA analysis was performed with multiple comparisons test (alpha=0.05) for FIG. 4b (Tukey multiple comparison test, *100 nM condition analyzed), FIG. 5 (100 nM evaluated, Tukey multiple comparison test), FIG. 9 and FIG. 10 (Dunnet multiple comparison test).

### Cytokine Analysis

Supernatants from FIGS. 1 and 5 were analyzed using the Millipore Sigma MILLIPLEX® MAP KIT (Burlington, Massachusetts), which measures human cytokines and chemokines. Supernatants were thawed on wet ice, centrifuged at 500×gravity (g) for 5 minutes at 4° C., then placed on ice. Samples were used at neat (no dilution) concentration. The magnetic bead panel kit was followed as per the manufacturer's protocol. A standard curve was prepared by serial dilution of the provided calibrator, reconstituted in RPMI media. A 25 μL aliquot of each sample or standard was added directly to the plates in technical duplicate. Subsequent incubations and washes were all carried out per manufacturer's protocol using a handheld magnet. Assay plates were read on the FLEXMAP 3D® System. Analysis was performed in the program FLEXMAP 3D® software to determine concentrations of cytokines per sample. Finally, data was placed in using Prism version 9 for visualization.

### Results

### Primary Bone Marrow Stromal Cells Reduce the Efficacy of a CD123×CD3 Antibody Against KG-1 Cells In Vitro

The bone marrow is composed of a heterogenous population of immune and stromal cells. One of the components of BM stromal cells are mesenchymal stem cells (MSCs) which can differentiate into fibroblasts, osteoblasts, and adipocytes. It has been shown previously that BM stromal fibroblasts can suppress T cell redirection-mediated cytotoxicity via cell-cell interactions implicating VLA-4 (Nair-Gupta P et al., Blockade of VLA4 sensitizes leukemic and myeloma tumor cells to CD3 redirection in the bone marrow microenvironment. Blood Cancer Journal. 2020; 10(6)). To test whether other components of the BM can reduce the cytotoxic effect of T cell recruiter antibodies, a CD123×CD3 bispecific antibody was tested against CD123⁺ AML cells in the presence of healthy control (HC) pan CD3⁺ T cells and various BM stromal cell types. (Nair-Gupta P et al., Blockade of VLA4 sensitizes leukemic and myeloma tumor cells to CD3 redirection in the bone marrow microenvironment. Blood Cancer Journal. 2020; 10(6); Gaudet F et al., Development of a CD123×CD3 Bispecific Antibody (JNJ-63709178) for the Treatment of Acute Myeloid Leukemia (AML). Blood. 2016; 128(22):2824) In the absence of stromal cells, the CD123×CD3 bispecific antibody killed KG-1 cells effectively (78% max cytotoxicity, CD123^{high}; bone marrow origin (FIG. 1)). In contrast, the presence of HS-5 stromal cells resulted in a significant decrease in killing of KG-1 cells (38-50%; FIG. 1 and Table 1). Similar results were observed with other BM stromal components such as primary BM stromal cells, mesenchymal stem cells, human osteoblasts and adipocytes suggesting that several components of the BM can suppress T cell-mediated cytotoxicity (FIG. 1 and Table 1). The KG-1 cell line was originally isolated from the BM of a patient with leukemia and may be more susceptible to the effects of stromal interactions (Mrozek K, et al., Molecular cytogenetic characterization of the KG-1 and KG-la acute myeloid leukemia cell lines by use of spectral karyotyping and fluorescence in situ hybridization. Genes Chromosomes Cancer. 2003; 38(3):249-252). To assess whether a CD123⁺ cell line which did not originate from the BM would be similarly affected by BM stromal cells, the impact of these stromal cells was evaluated in the presence of HL-60 cells (CD123^{low}), which originate from peripheral blood of a promyelocytic leukemia and which may be potentially less dependent on stromal interactions (Collins S J et al., Terminal differentiation of human promyelocytic leukemia cells induced by dimethyl sulfoxide and other polar compounds. Proc Natl Acad Sci USA. 1978; 75(5):2458-2462). Indeed, while the CD123×CD3 bispecific antibody was able to kill HL-60 cells, the BM stromal cells did not significantly impact the cytotoxic activity as much (5-22% reduction; Table 2 and Table 3). Interestingly, T cell activation (as evaluated using the CD25 cell surface marker) was only moderately affected in KG-1 cells when in co-culture with stromal cells (except osteoblasts) while HL-60 cells were not significantly altered (FIG. 1 and Table 1). The reduced expression of CD25 observed in the presence of stromal cells also correlated with reduced cytokine secretion (FIGS. 2 and 3). These results suggest that the presence of stromal cells can suppress T cell function leading to reduced killing of target cells.

TABLE 1
Stromal impact on EC₅₀ values and percent maximum cytotoxicity

| Target Cell | Stroma | EC₅₀ (nM) | % Max |
|---|---|---|---|
| KG-1 | NS | 0.0004 | 78 |
| KG-1 | HS-5 | 0.0073 | 39 |
| KG-1 | BM | 0.0157 | 28 |
| KG-1 | MSC | 0.0079 | 40 |
| KG-1 | Osteoblasts | 0.0053 | 37 |
| KG-1 | Adipocytes | 0.0071 | 37 |
| HL-60 | NS | 0.2144 | 73 |
| HL-60 | HS-5 | 0.1300 | 51 |
| HL-60 | BM | 0.1037 | 58 |
| HL-60 | MSC | 0.1306 | 68 |
| HL-60 | Osteoblasts | 0.1098 | 64 |
| HL-60 | Adipocytes | 0.1880 | 68 |

EC, Effective Concentration; NS, no stromal cell added; BM, bone marrow stromal cells; MSC, mesenchymal stem cells; EC₅₀ values are from 48-hour timepoint; and % Max represents Top value from 4-parameter non-linear fit curve.

TABLE 2
Impact of anti-CD44 to EC₅₀ values
and percent maximum cytotoxicity

| Target Cell | Stroma | Anti-CD44 | EC₅₀ (nM) | % Max |
|---|---|---|---|---|
| KG-1 | NS | − | 0.0028 | 95 |
| KG-1 | HS-5 | − | 0.0319 | 52 |
| KG-1 | NS | + | 0.0009 | 94 |
| KG-1 | HS-5 | + | 0.0265 | 91 |
| HL-60 | NS | − | 0.0868 | 86 |
| HL-60 | HS-5 | − | 0.1419 | 72 |
| HL-60 | NS | + | 0.0808 | 84 |
| HL-60 | HS-5 | + | 0.0762 | 84 |
| MV4-11 | NS | − | 0.0056 | 93 |
| MV4-11 | HS-5 | − | 0.0057 | 33 |
| MV4-11 | NS | + | 0.0026 | 92 |
| MV4-11 | HS-5 | + | 0.0025 | 66 |
| MOLM-13 | NS | − | 0.0362 | 81 |
| MOLM-13 | HS-5 | − | 0.0262 | 18 |
| MOLM-13 | NS | + | 0.0125 | 63 |
| MOLM-13 | HS-5 | + | 0.1533 | 56 |
| SKNO-1 | NS | − | 0.0111 | 62 |
| SKNO-1 | HS-5 | − | 0.0757 | 25 |
| SKNO-1 | NS | + | 0.0077 | 76 |
| SKNO-1 | HS-5 | + | 0.1085 | 44 |

EC, effective concentration; NS, no stroma.
EC50 values are from 48 hour timepoint and % Max represents Top value from 4-parameter non-linear fit curve.

TABLE 3
Statistical analysis of HS-5 mediated suppression of CD123 ×
CD3 cytotoxicity and restoration with anti-CD44

| Comparison | Assay | KG-1 | HL-60 | MV4-11 | MOLM-13 | SKNO-1 |
|---|---|---|---|---|---|---|
| NS no ab vs. HS-5 no ab | Cytotoxicity | 0.0001 | 0.0006 | <0.0001 | <0.0001 | 0.0003 |
| NS no ab vs. NS anti-CD44 | Cytotoxicity | 0.9762 | 0.4002 | 0.9998 | 0.0026 | 0.0076 |
| HS-5 no ab vs. HS-5 anti- | Cytotoxicity | 0.0002 | 0.0011 | 0.0003 | <0.0001 | 0.0019 |
| CN44 | | | | | | |
| NS no ab vs. HS-5 no ab | CD25 | 0.4024 | <0.0001 | 0.9613 | 0.2339 | 0.0258 |
| NS no ab vs. NS anti-CD44 | CD25 | 0.9837 | 0.5585 | >0.9999 | 0.9996 | 0.9997 |
| HS-5 no ab vs. HS-5 anti- | CD25 | 0.5754 | <0.0001 | 0.4713 | 0.3845 | 0.2333 |
| CD44 | | | | | | |
| NS no ab vs. HS-5 no ab | PD-1 | 0.0135 | 0.0523 | 0.0008 | 0.0098 | 0.0778 |
| NS no ab vs. NS anti-CD44 | PD-1 | 0.9874 | 0.9915 | 0.9100 | 0.9974 | 0.9668 |
| HS-5 no ab vs. HS-5 anti- | PD-1 | 0.1201 | 0.0456 | 0.2631 | 0.3600 | 0.7451 |
| CD44 | | | | | | |

P Values displayed above are a representation of statistical significance of either HS-5 mediated suppression of CD123 × CD3 cytotoxicity or T cell activation (CD25 or PD-1) and restorative effect of anti-CD44 addition to each respective AML cell line. One-way ANOVA with a Tukey multiple comparison test of CD123 × CD3 (100 nM) evaluated (alpha = 0.05). P values > 0.05, not significantly different.
Ab, antibody; NS, no stromal cells.

### CD44 is Upregulated on AML Cells

It has been shown that VLA-4 could mediate interactions between tumor cells and stromal cells (fibroblasts) leading to T cell suppression and that blockade of VLA-4 could restore killing from T cell redirection agents (Nair-Gupta P et al., Blockade of VLA4 sensitizes leukemic and myeloma tumor cells to CD3 redirection in the bone marrow microenvironment. Blood Cancer Journal. 2020; 10(6)). To identify additional proteins that could mediate this suppression, various candidate proteins implicated in cell-cell surface interactions in AML were considered. One particular protein, CD44, has been shown to contribute to the adherent properties of HSCs and LSCs (Schroeder T et al., Mesenchymal stromal cells in myeloid malignancies. Blood Res. 2016; 51(4):225-232; Pleyer L et al., Mesenchymal Stem and Progenitor Cells in Normal and Dysplastic Hematopoiesis-Masters of Survival and Clonality. Int J Mol Sci. 2016; 17(7)). In AML, CD44 is upregulated on AML blasts and LSCs and functions as a survival signal through a variety of pathways (Gul-Uludag H et al., Polymeric nanoparticle-mediated silencing of CD44 receptor in CD34+ acute myeloid leukemia cells. Leukemia research. 2014; 38(11): 1299-1308; Garg S et al., Antigen expression on a putative leukemic stem cell population and AML blast. Int J Hematol. 2016; 103(5):567-571; Chen P et al., Bone marrow stromal cells protect acute myeloid leukemia cells from anti-CD44 therapy partly through regulating PI3K/Akt-p27(Kip1) axis. Mol Carcinog. 2015; 54(12):1678-1685; and Gadhoum S Z et al., Anti-CD44 antibodies inhibit both mTORC1 and mTORC2: a new rationale supporting CD44-induced AML differentiation therapy. Leukemia. 2016; 30(12):2397-2401). CD44 protein expression in various AML cell lines was evaluated and found to be consistently expressed (FIG. 4A). Next, CD44 mRNA expression in primary AML cells (n=202) was assessed and a significant upregulation compared to myelodysplastic syndrome (MDS; n=164) and normal control (n=69; FIG. 4B) was found (Mills K I et al., Microarray-based classifiers and prognosis models identify subgroups with distinct clinical outcomes and high risk of AML transformation of myelodysplastic syndrome. Blood. 2009; 114(5): 1063-1072). This is consistent with the idea that CD44 expression may be important in disease progression from MDS to AML disease.

### Anti-CD44 Restores Stromal-Mediated Suppression of Killing by CD123×CD3

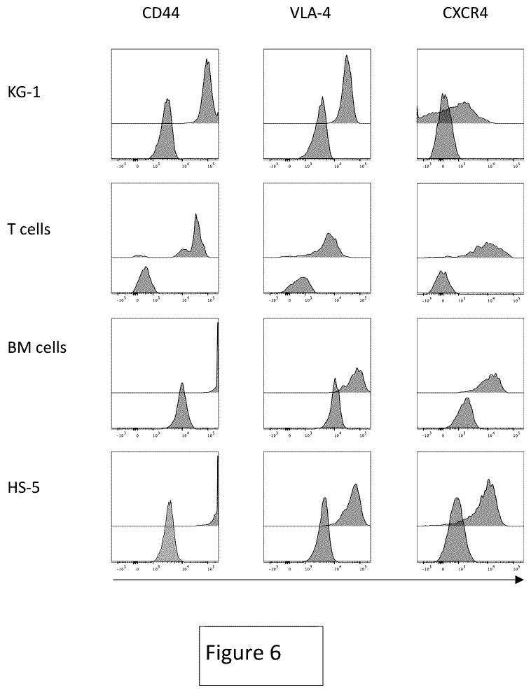

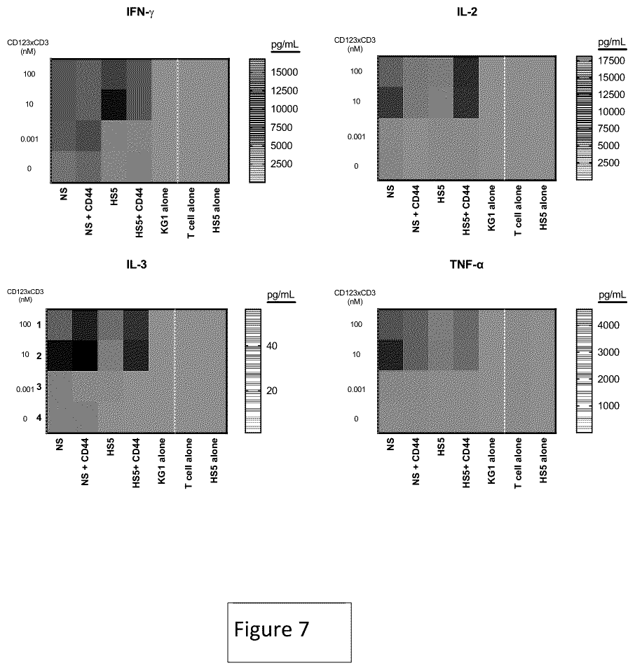

To evaluate whether CD44 is implicated in the stromal-mediated suppression of T cell killing by CD123×CD3 bispecific antibody, an anti-CD44 antibody was added to the co-cultures to neutralize CD44. Additional AML cell lines (MV4-11, MOLM-13, and SKNO-1; all CD123⁺) were also tested. As shown in FIG. 5, the CD123×CD3 bispecific antibody resulted in robust and significant cytotoxic activity among all cell lines tested in the absence of stromal cells (Tables 3 and 4). In this context, the anti-CD44 antibody had no impact on the cytotoxic activity or T cell activation. In contrast, when AML cell lines were co-cultured with stromal cells, the anti-CD44 antibodies, the cytotoxic effect and T cell activation was restored to various degrees in all the cell lines tested except for the HL-60 cells which is CD44^{low} (FIG. 5). All cells tested were shown to express CD44 (FIG. 6). These results further confirm that the previous findings with other CD123⁺ AML cells suggest that the role of CD44 in AML may be broader. Cytokine were further evaluated in the supernatants and found to correlate with cytotoxicity. Specifically, addition of the CD123×CD3 bispecific antibody in the absence of stroma resulted in potent release of IFN-γ, IL-2, IL-3, and TNF-α at the two highest concentrations of CD123×CD3 bispecific antibody tested; however, addition of HS-5 cells blunted the cytokine release. The addition of anti-CD44 resulted in similar levels of IFN-γ, IL-3, and TNF-α but reduced IL-2 levels to near background. When anti-CD44 was added to the co-cultures containing HS-5 cells, IFN-γ levels were fully restored, while, IL-2, IL-3, and TNF-α levels were partially restored (FIG. 7). In aggregate, these results suggest that CD44 expression can reduce the sensitivity to T cell redirection therapeutics in the presence of stromal cells and that blockade of CD44 with an antibody can restore this activity.

TABLE 4
Statistical Analysis of Stromal Cells vs No Stromal Cells
on CD123 × CD3 Cytotoxicity against KG-1 and HL-60 cells

| Target | | | | | Osteo- | |
|---|---|---|---|---|---|---|
| Cells | Assay | HS-5 | BM | MSC | blast | Adipocyte |
| KG-1 | Cytotoxicity | <0.0001 | 0.0197 | 0.0010 | 0.0003 | 0.0004 |
| HL-60 | Cytotoxicity | 0.0081 | 0.1006 | 0.6052 | 0.3462 | 0.5896 |
| KG-1 | CD25 | 0.0614 | 0.0022 | 0.1098 | 0.0006 | 0.4949 |
| HL-60 | CD25 | 0.5298 | 0.0786 | 0.5868 | 0.5927 | 0.6147 |

P values displayed above as a representation of statistical significance for CD123 × CD3 cell cytotoxicity comparing respective stromal cells to no stromal cell condition. Extra sum-of-squares F test analysis was performed within a 4-parameter nonlinear curve fit by comparing the top parameter (alpha = 0.05). P values > 0.05, not significantly different.
BM, bone marrow stromal cells; MSC, mesenchymal stem cells.

### CD44 KO Cell Lines are Resistant to HS-5 Stromal-Mediated Suppression of CD123×CD3

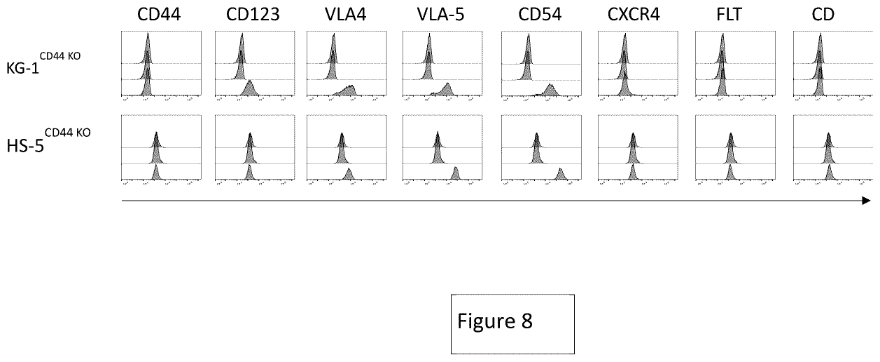

Since addition of anti-CD44 antibodies to the co-cultures restored the function of CD123×CD3 bispecific antibody, it was hypothesized that CD44 is playing an important role in the suppression of T cell-mediated killing by CD123×CD3 bispecific antibody. CD44 is expressed on HS-5 stromal cells and KG-1 AML cells. To establish whether the impact of the anti-CD44 antibody originates from the blocking of CD44 on stromal cells or tumor cells (or both), a KO of CD44 in HS-5 cells and in tumor cells were generated. As shown in FIG. 8, cells were successfully generated that no longer express CD44 (while the levels of control VLA-4 and CXCR4 remained unchanged). These cells were then used in cytotoxicity assays in the presence of CD123×CD3 bispecific antibody to assess the impact of the loss of CD44 on the surface of either cell type. FIG. 9 shows that cytotoxicity was significantly restored when HS-5CD44KO or KG-1CD44KO cells (Table 5) were used in the co-culture system. The data suggest that the impact was similar whether CD44 was deleted in tumor cells or stromal cells. Deletion of CD44 in both cell types did not result in any additive outcome. This data argues that CD44 expressed on both HS-5 and KG-1 can contribute to the suppressive effect seen with HS-5 cells and deletion of CD44 on either cell type can restore cytotoxic function of the CD123×CD3 bispecific antibody. These findings are consistent with the experiments using anti-CD44 described above and further establish CD44 expression as a resistant mechanism to T cell redirection in AML.

TABLE 5
Statistical analysis of CD123 × CD3-mediated cytotoxicity,
T cell activation (CD25 and PD-1), and granzyme b levels
with either anti-CD44 and/or CD44 KO cell lines versus KG-1
cells co-cultured with HS-5^{wt} cells

| | Cyto- | | Granzyme | |
|---|---|---|---|---|
| KG-1 + HS-5 vs. | toxicity | CD25 | B | PD-1 |
| KG-1 | <0.0001 | 0.0051 | 0.0043 | 0.0769 |
| KG-1^{KO} | <0.0001 | 0.0148 | 0.2292 | 0.0240 |
| KG-1 + HS-5^{KO} | <0.0001 | 0.0157 | 0.5552 | 0.9073 |
| KG-1^{KO} + HS-5 | 0.0074 | 0.7102 | 0.9934 | 0.4905 |
| KG-1^{KO} + HS-5^{KO} | <0.0001 | 0.0346 | 0.0136 | 0.6101 |
| KG-1 + anti-CD44 | <0.0001 | 0.2243 | 0.9997 | 0.4871 |
| KG-1^{KO} + anti-CD44 | <0.0001 | 0.9999 | 0.0002 | 0.3294 |
| KG-1 + HS-5 + anti-CD44 | <0.0001 | 0.0922 | 0.6740 | 0.9995 |
| KG-1 + HS-5^{KO} + anti-CD44 | 0.0010 | 0.1954 | 0.3638 | 0.9991 |
| KG-1^{KO} + HS-5 + anti-CD44 | <0.0001 | 0.0071 | 0.0151 | 0.9993 |
| KG-1^{KO} + HS-5^{KO} + anti-CD44 | <0.0001 | 0.8543 | 0.0046 | 0.9073 |

P values displayed above are a representation of statistical significance of each cytokine for its respective comparison. One-way ANOVA with a Dunnet multiple comparison test of CD123 × CD3 evaluated (alpha = 0.05).
P values > 0.05, not significantly different.
KO, knockout;
wt, wildtype.

### Blockade of CD44 Enhances Granzyme-B Levels and Increases PD-1 Expression

To establish whether neutralization or ablation of CD44 on tumor cells or stromal cells directly impacted T cell function, it was decided to determine the levels of granzyme-B in T cells which are secreted in the synapse by the T cells and taken by the tumor cells, resulting in induction of apoptosis. As shown in FIG. 10 and Table 5, the addition of anti-CD44 antibody or KO of CD44 in KG-1 or HS-5 cells resulted in the increase of granzyme-B in T cells, which could partly explain the greater cytotoxicity. Surprisingly, the addition of anti-CD44 also resulted in increased granzyme-B when T cells were incubated with KG-1CD44 KO cells. Similar results were found when assessing PD-1 expression on T cells (FIG. 10 and Table 5). These results suggest that CD44 expression may suppress cytotoxicity of T cell redirection antibodies through mechanisms other than expression on the tumor cells.

### DISCUSSION

Minimal residual disease following primary chemotherapy or autologous hematopoietic stem cell transplant in AML is the primary reason for relapse. Since the BM is a protective microenvironment for AML LSCs, these cancer cells are generally resistant to secondary therapies and treatments. Understanding the mechanisms of resistance in the context of the BM microenvironment is crucial for current and future therapies including immune redirection approaches.

It is shown herein that multiple BM stromal cell types can inhibit the activity of a T cell redirector antibody, the CD123×CD3 bispecific antibody, against KG-1 cells. While T cell activation (CD25 expression) was less affected; cytokine release was potently suppressed by primary BM stromal cells, which correlated with the cytotoxicity.

The results argue that CD44 expression on BM stromal cells and tumor cells plays an important role in BM stromal suppression of the cytotoxic activity of the CD123×CD3 bispecific antibody. Inhibition of CD44 by an anti-CD44 antibody or KO of the CD44 gene by CRISPR in either tumor cells or stromal cells rescued to various degrees the stromal-mediated suppression depending on the cell line used. The inhibition of CD44 not only restored the cytotoxicity of our T cell redirection agent, the CD123×CD3 bispecific antibody, but also caused an increase in T cell activation and cytokine release, consistent with tumor cell killing.

1. A pharmaceutical composition comprising a T cell redirection therapeutic and an anti-CD44 therapeutic, wherein, the T cell redirection therapeutic comprises a first binding region that immunospecifically binds a T cell surface antigen and a second binding region that immunospecifically binds a tumor associated antigen (TAA).

2. The pharmaceutical composition of claim 1, further comprising a pharmaceutically acceptable carrier.

3. The pharmaceutical composition of claim 1, wherein the T cell redirection therapeutic is an antibody or antigen-binding fragment thereof.

4. The pharmaceutical composition of claim 1, wherein the T cell surface antigen is selected from the group consisting of CD3, CD2, CD4, CD5, CD6, CD8, CD28, CD40L, CD44, KI2L4, NKG2E, NKG2D, NKG2F, BTNL3, CD186, BTNL8, PD-1, CD195, and NKG2C.

5. The pharmaceutical composition of claim 4, wherein the T cell surface antigen is CD3.

6. The pharmaceutical composition of claim 1, wherein the TAA is selected from the group consisting of CD123, BCMA, GPRC5D, CD33, CD19, PSMA, TMEFF2, CD20, CD22, CD25, CD52, ROR1, HM1.24, CD38, and SLAMF7.

7. The pharmaceutical composition of claim 6, wherein the T cell redirection therapeutic is a CD123×CD3 bispecific antibody having a first antigen-binding site that immunospecifically binds CD123 and a second antigen-binding site that immunospecifically binds CD3.

8. The pharmaceutical composition of claim 7, wherein the CD123×CD3 bispecific antibody comprises a first heavy chain (HC1), a first light chain (LC1), a second heavy chain (HC2), and a second light chain (LC2), and wherein the HC1 and the LC1 pair to form the first antigen-binding site and the HC2 and the LC2 pair to form the second antigen-binding site.

9. The pharmaceutical composition of claim 8, wherein the HC1 comprises the amino acid sequence of SEQ ID NO: 1, the LC1 comprises the amino acid sequence of SEQ ID NO: 2, the HC2 comprises the amino acid sequence of SEQ ID NO: 3, and the LC2 comprises the amino acid sequence of SEQ ID NO: 4.

10. The pharmaceutical composition of claim 6, wherein the T cell redirection therapeutic is a BCMA×CD3 bispecific antibody having a first antigen-binding site that immunospecifically binds CD123 and a second antigen-binding site that immunospecifically binds CD3.

11. The pharmaceutical composition of claim 10, wherein the BCMA×CD3 bispecific antibody comprises a first heavy chain (HC1), a first light chain (LC1), a second heavy chain (HC2), and a second light chain (LC2), and wherein the HC1 and the LC1 pair to form the first antigen-binding site and the HC2 and the LC2 pair to form the second antigen-binding site.

12. The pharmaceutical composition of claim 11, wherein the HC1 comprises the amino acid sequence of SEQ ID NO: 5, the LC1 comprises the amino acid sequence of SEQ ID NO: 6, the HC2 comprises the amino acid sequence of SEQ ID NO: 7, and the LC2 comprises the amino acid sequence of SEQ ID NO: 8.

13. The pharmaceutical composition of claim 1, wherein the anti-CD44 therapeutic is an anti-CD44 antibody or antigen-binding fragment thereof.

14. The pharmaceutical composition of claim 13, wherein the anti-CD44 antibody or antigen-binding fragment thereof is selected from the group consisting of monoclonal antibodies, scFv, Fab, Fab′, F(ab′)2, and F(v) fragments, heavy chain monomers or dimers, light chain monomers or dimers, and dimers consisting of one heavy chain and one light chain.

15. The pharmaceutical composition of claim 1, wherein the anti-CD44 therapeutic is a CD44 antagonist.

16. The pharmaceutical composition of claim 1, further comprising a VLA-4 adhesion pathway inhibitor.

17. The pharmaceutical composition of claim 16, wherein the VLA-4 adhesion pathway inhibitor is an anti-VLA-4 antibody or antigen-binding fragment thereof.

18. The pharmaceutical composition of claim 17, wherein the anti-VLA-4 antibody or antigen-binding fragment thereof is selected from the group consisting of monoclonal antibodies, scFv, Fab, Fab′, F(ab′)2, and F(v) fragments, heavy chain monomers or dimers, light chain monomers or dimers, and dimers consisting of one heavy chain and one light chain.

19. The pharmaceutical composition of claim 16, wherein the VLA-4 adhesion pathway inhibitor is a VLA-4 antagonist.

20. The pharmaceutical composition of claim 19, wherein the VLA-4 antagonist is selected from the group consisting of BIO1211, TCS2314, BIO5192, and TR14035.

21. A method of killing cancer cells, comprising disrupting cell-cell contact between cancer cells and stromal cells comprising subjecting cancer cells to therapeutically effective amounts of the pharmaceutical composition of claim 1.

22. The method of claim 20, wherein the cancer is a hematological malignancy or a solid tumor.

23. The method of claim 22, wherein the cancer is AML or MM.

24. The method of claim 21, wherein the T cell redirection therapeutic and the anti-CD44 therapeutic are administered simultaneously or sequentially.

25. The method of claim 24, wherein the anti-CD44 therapeutic is administered prior to administration of the T cell redirection therapeutic.

26. The method of claim 24, wherein the anti-CD44 therapeutic is administered after administration of the T cell redirection therapeutic.

27. A kit comprising the pharmaceutical composition of claim 1.


SEQUENCE LISTING (8 sequences)

SEQ ID NO: 1 - protein, 445 aa, Artificial Sequence
  Synthetic peptide
EVQLVQSGAEVKKPGESLKISCKGSGYSFTSYWISWVRQMPGKGLEWMGIIDPSDSDTRYSPSFQGQVTISADKSISTAYLQWSSLKASDTAMYYCARGDGSTDLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 215 aa, Artificial Sequence
  Synthetic peptide
EIVLTQSPGTLSLSPGERATLSCRASQSVSSSYLAWYQQKPGQAPRLLIYGASSRATGIPDRFSGSGSGTDFTLTISRLEPEDFAVYYCQQDYGFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 452 aa, Artificial Sequence
  Synthetic peptide
EVQLVESGGGLVQPGGSLKLSCAASGFTFNTYAMNWVRQASGKGLEWVGRIRSKYNAYATYYAASVKGRFTISRDDSKNTAYLQMNSLKTEDTAVYYCTRHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 4 - protein, 215 aa, Artificial Sequence
  Synthetic peptide
QAVVTQEPSLTVSPGGTVTLTCRSSTGAVTTSNYANWVQQKPGQAPRGLIGGTNKRAPGTPARFSGSLLGGKAALTLSGAQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 5 - protein, 448 aa, Artificial Sequence
  Synthetic peptide
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 214 aa, Artificial Sequence
  Synthetic peptide
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 7 - protein, 452 aa, Artificial Sequence
  Synthetic peptide
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 8 - protein, 215 aa, Artificial Sequence
  Synthetic peptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS